(19) United States
(12) Patent Application Publication
(10) Pub. No.: US 2018/0316781 A1
(43) Pub. Date: Nov. 1, 2018

(54) SYSTEM FOR REMOTE NONINVASIVE CONTACTLESS ASSESSMENT AND PREDICTION OF BODY ORGAN HEALTH

(71) Applicant: Mores, Inc., Burbank, CA (US)

(72) Inventor: Ayman Salem, Burbank, CA (US)

(73) Assignee: Mores, Inc.

(21) Appl. No.: 16/027,352

(22) Filed: Jul. 4, 2018

Related U.S. Application Data
(63) Continuation-in-part of application No. 15/943,585, filed on Apr. 2, 2018.
Application No. 15/943,585 is a continuation-in-part of application No. 15/797,650, filed on Oct. 30, 2017.
Application No. 15/797,650 is a continuation of application No. 14/952,424, filed on Nov. 25, 2015, now Pat. No. 9,838,508.
Application No. 14/952,424 is a continuation-in-part of application No. 14/080,021, filed on Nov. 14, 2013, now Pat. No. 9,747,417.

(51) Int. Cl. H04L 29/06; H04L 29/08; G16H 50/20; G16H 10/60; G16H 15/00; G16H 10/40; G16H 40/63

(52) U.S. Cl. CPC H04L 67/42; H04L 67/06; G16H 50/20; G16H 40/63; G16H 15/00; G16H 10/40; G16H 10/60

(57) ABSTRACT

A noninvasive patient internal bodily assessment design is provided. The design includes a personal care display device having a plurality of sensors configured to sense attributes of a patient, wherein the plurality of sensors includes a video image collection device and an audio collection device, and a remote central server device arrangement connected to the personal care device. The personal care display device is configured to receive information from the patient, including receiving multiple pieces of information from the video image collection device from the exterior of the patient, compile information about the patient and determine organ health or other relevant health attributes of the patient based at least in part on the multiple pieces of information received from the video image collection device.

The present application is a continuation-in-part of co-pending U.S. patent application Ser. No. 15/943,585, entitled “Enhanced Personal Care System Employing Blockchain Functionality,” inventor Ayman Salem, filed Apr. 2, 2018, which is a continuation-in-part of co-pending U.S. patent application Ser. No. 15/797,650, entitled “Method and Apparatus for Enhanced Personal Care with Interactive Diary,” inventor Ayman Salem, filed Oct. 30, 2017, which is a continuation of co-pending U.S. patent application Ser. No. 14/952,424, entitled “Method and Apparatus for Enhanced Personal Care with Interactive Diary,” inventor Ayman Salem, filed Nov. 25, 2015, now U.S. Pat. No. 9,838,508, which is a continuation-in-part of co-pending U.S. patent application Ser. No. 14/080,021, inventor Ayman Salem, entitled “Method and Apparatus for Enhanced Personal Care,” filed Nov. 14, 2013, now U.S. Pat. No. 9,747,417, the entirety of which are incorporated herein by reference.

### BACKGROUND OF THE INVENTION

### Field of the Invention

The present invention relates generally to health care, and more specifically to systems and methods of noninvasively assessing patient health, including organ health.

### Description of the Related Art

Obtaining vital signs of bodily elements, such as body organs, has been established in the art using direct non-invasive single source measurements, such as listening to the sound of a heartbeat using a stethoscope to assess heart rate and heart health. Doctors measure elevation of a mercury column, for example, to estimate the pressure needed to occlude blood flow in an artery and estimate systolic and diastolic blood pressures using doppler ultrasound in different modes to estimate cardiac output.

However, certain measurements of bodily fitness, including organ fitness, are unavailable to even the most modern physicians without the need to resort to invasive procedures. Invasive procedures are of course undesirable and sometimes difficult to perform from a remote location, such as during a tele-health session. Physicians are thus left to forgo bodily organ examination altogether or rely on infrequent invasive laboratory biochemical work-up. For example, physicians can have blood drawn from the patient and can run a series of blood tests to determine the current state of a patient's organs, but such measurements can be infrequent and/such patient attributes can change rapidly, leading to imprecise health assessments. Indeed, certain measurements taken under stressful conditions, such as in the case of a patient particularly concerned about being in a doctor's office, can result in inaccurate measurements and could potentially be detrimental to the patient.

It would be therefore beneficial to provide a system that enhances the ability to noninvasively and remotely assess, validate, and predict vital signs of a specific individual patient in a way that overcomes the issues associated with invasive medical assessment procedures.

### SUMMARY OF THE INVENTION

Thus according to one aspect of the present design, there is provided an apparatus comprising a sensor configured to optically detect blood distribution analysis and spread at least two external regions of a user via at least one of skin color, skin tone, skin temperature, skin condition, and capillary refill, and a processor configured to determine an image based on the optical detection of the sensor, the image indicating a first blood distribution and spread for a first region of the user and a second image indicating a second blood distribution and spread for a second region. The processor compares the first blood distribution and spread for the first region and the second blood distribution and spread for the second region to provide a blood-distribution/spread asymmetry representing a difference in heart health between the first region and the second region.

According to a further embodiment, there is provided an apparatus comprising a microphone configured to receive audio data including body organs sounds of a patient at a first point of the patient's body, wherein the audio data comprises capillary refill audio data, and a processor configured to compare the audio data received with audio data previously recorded at the first point of the patient's body and with different data obtained from a different non-audio sensor and determine at least one of respiratory rate (RR), lung volume, pulse oximetry, breathing characteristics, jugular vein distension (JVD), and/or body pH level, pulse/heart rate, blood pressure, heart rhythm, and EKG values.

According to another embodiment, there is provided an apparatus comprising a personal care display device comprising a plurality of sensors configured to sense attributes of a patient, wherein the plurality of sensors comprises a video image collection device and an audio collection device, and a remote central server device arrangement connected to the personal care device. The personal care display device is configured to receive information from the patient, including receiving multiple pieces of information from the video image collection device from the exterior of the patient, compile information about the patient, such as in a 2D, 3D or holographic representation, and determine organ health of the patient based at least in part on the multiple pieces of information received, collected, or gathered from sensors, such as multiple sensors including audio sensor(s) and video image collection device(s).

These and other advantages of the present invention will become apparent to those skilled in the art from the following detailed description of the invention and the accompanying drawings.

### BRIEF DESCRIPTION OF THE DRAWINGS

For a more complete understanding of the present disclosure, reference is now made to the following figures, wherein like reference numbers refer to similar items throughout the figures:

FIG. 1 is a general system overview of one aspect of the present design;

FIG. 2A illustrates a rectangular embodiment of one aspect of the present design;

FIG. 2B illustrates an oval embodiment according to another aspect of the present design;

FIG. 3 is a general representation of certain components employed in the present design;

FIG. 4A illustrates a side view of a device similar to that shown in FIG. 2;

FIG. 4B is a rear view of an embodiment of the device;

FIGS. 5A-5C illustrate apparatus including a set of separate USB receptacles that may be provided as an alternative to or in addition to the receptacles or slots provided on the device;

FIG. 6 illustrates an example of a magnification camera that may be employed with or provided as a module;

FIG. 7 illustrates an example of a sensing bracelet that may be employed with or provided as a module in accordance with the present design;

FIG. 8 illustrates a tape measure that may be employed as or in accordance with a module in the present design;

FIG. 9 is a reflective band or strap 901 that may be employed in association with a module;

FIG. 10 shows a spirometer arrangement that may be employed as part of the present design;

FIG. 11 illustrates a portable scale that may be used with the present design;

FIG. 12 is a pill dispenser that may be employed with the present design;

FIG. 13 shows a representative caliper that may be used with the present design;

FIG. 14 illustrates a handheld unit that may be used with the present design;

FIG. 15 is a simple functional example/flow diagram of use of a scale module;

FIG. 16 illustrates an appearance of a screen operating in accordance with the interactive diary function;

FIG. 17 shows one embodiment of general operation of the interactive diary function;

FIG. 18 generally illustrates the overall concept disclosed herein with respect to disease points and health points and the distribution of such points;

FIG. 19 represents the system employing blockchain technology and a recommender system for use in bidding and forming agreements for disease points and health points; and

FIG. 20 illustrates general operation of the telehealth/noninvasive bodily assessment aspect of the present design.

The exemplification set out herein illustrates particular embodiments, and such exemplification is not intended to be construed as limiting in any manner.

### DETAILED DESCRIPTION OF THE INVENTION

The following description and the drawings illustrate specific embodiments sufficiently to enable those skilled in the art to practice the system and method described. Other embodiments may incorporate structural, logical, process and other changes. Examples merely typify possible variations. Individual components and functions are generally optional unless explicitly required, and the sequence of operations may vary. Portions and features of some embodiments may be included in or substituted for those of others.

The following description and the drawings illustrate specific embodiments sufficiently to enable those skilled in the art to practice the system and method described.

Other embodiments may incorporate structural, logical, process and other changes. Examples merely typify possible variations. Individual components and functions are generally optional unless explicitly required, and the sequence of operations may vary. Portions and features of some embodiments may be included in or substituted for those of others.

In general, the present design is broadly directed to a system that employs hardware configured to noninvasively assess health attributes of a specific user or patient. The present design may take the form of a device including a reflective surface and at least one sensor and in certain instances more than one sensor, such as a camera and an audio collection device, where the device has processing and communication capability. The device may be configured with connection points, the connection points allowing personal care modules or medical assessment devices (blood pressure cuff, etc.) to be connected to the device. Additional functionality may be provided via a central or main hardware component. In this manner, an individual can maintain a device at her residence or place of work or elsewhere that can be configured to meet her personal care needs, including medical needs, and can provide noninvasive assessments of patients using the hardware provided. The user/patient may also enter information manually on the device.

In one embodiment, information received may include bowel sounds and sounds emanating from joints and other body parts. Such sounds may be assessed and recommendations or suggestions presented. In such an embodiment, the apparatus comprises a sensor configured to receive spectral gas data including body organs spectral gas data emanating from the mouth, bowel or sweat glands of a patient.

The system may include a portable device configured to be taken with the user/patient, while traveling locally or long distance, and the portable device may connect with the base device such that the user may record desired information and provide the information to the central location at a later time.

Additionally, the present system may include a communications network, which may be server or cloud based, wherein the user/patient can store personal care information, obtain personal care information such as recommendations specific to the user, may schedule and receive reminders, and so forth.

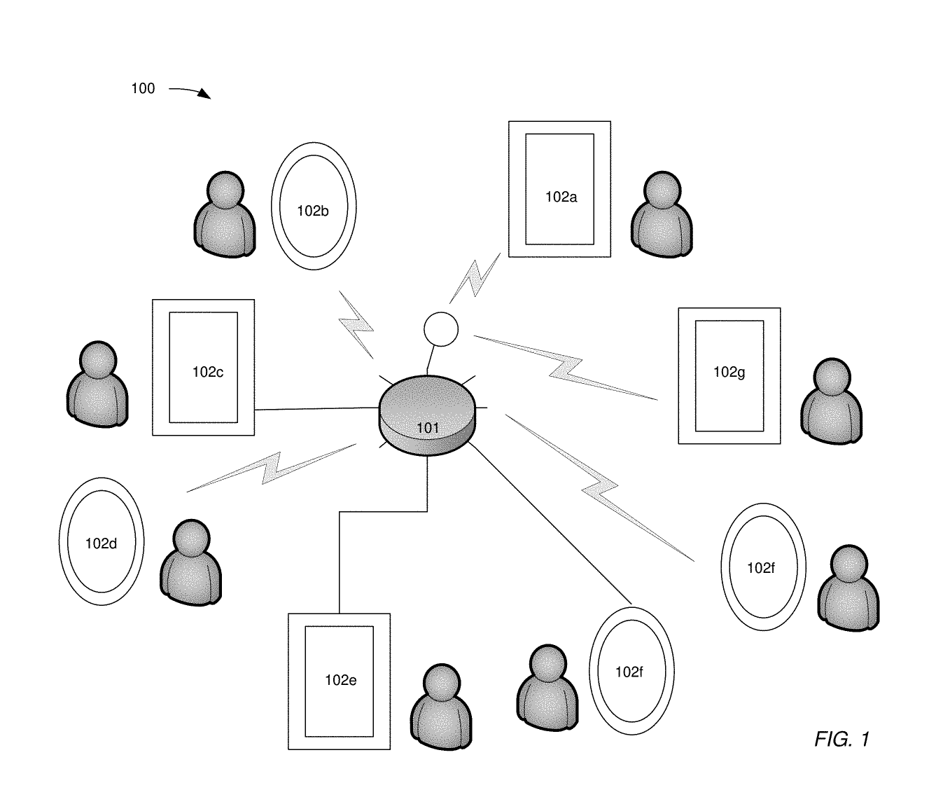

FIG. 1 is a general system overview of one aspect of the present design. From FIG. 1, network 100 includes a processing and storage site 101, also referred to as a remote device arrangement herein, configured to communicate with and receive communications from personal care devices 102a-h. Communication may be effectuated between the processing and storage site 101 and the personal care devices 102a-h in any manner known and available, including but not limited to internet connectivity via wire, wireless (802.11a/b/n/g, Wi fi), cellular, and or other communication means. While a single processing and storage site 101 is shown, it is to be understood that more than one such site may be employed and more than one such site may carry out some of the functionality disclosed herein.

In general, information about an individual's personal care may be obtained at a personal care device, e.g. personal care device 102b, and this information may be communicated to processing and storage site 101. Information about personal care generally falls into five categories, but other categories may be employed or provided, the five categories including health, fitness/wellness, fashion, cosmetics, and pharmaceuticals. While personal care device 102b may offer such capabilities, a user may or may not employ all capabilities. A user may simply wish to employ the present design for health purposes. Information obtained may be manually entered by the user or obtained using a set of readings or information obtained at the device, and/or information may be provided to the personal care device 102b from the processing and storage site. As an example, blood pressure readings may be made by the user using the personal care device, the blood pressure information may be provided from the personal care device 102b to the processing and storage site 101, and the processing and storage site 101 may provide a recommendation back to the user, even something as innocuous as “You have a slightly elevated blood pressure. The following may be potential meal suggestions for the coming days.” As may be understood, any type of interaction between the user, user device 102b, and processing and storage site 101 may be employed as desirable, such as determining the user's body type and recommending clothing for the user, determining skin tone and making a recommendation for particular skin care and/or cosmetic purchases, and so forth.

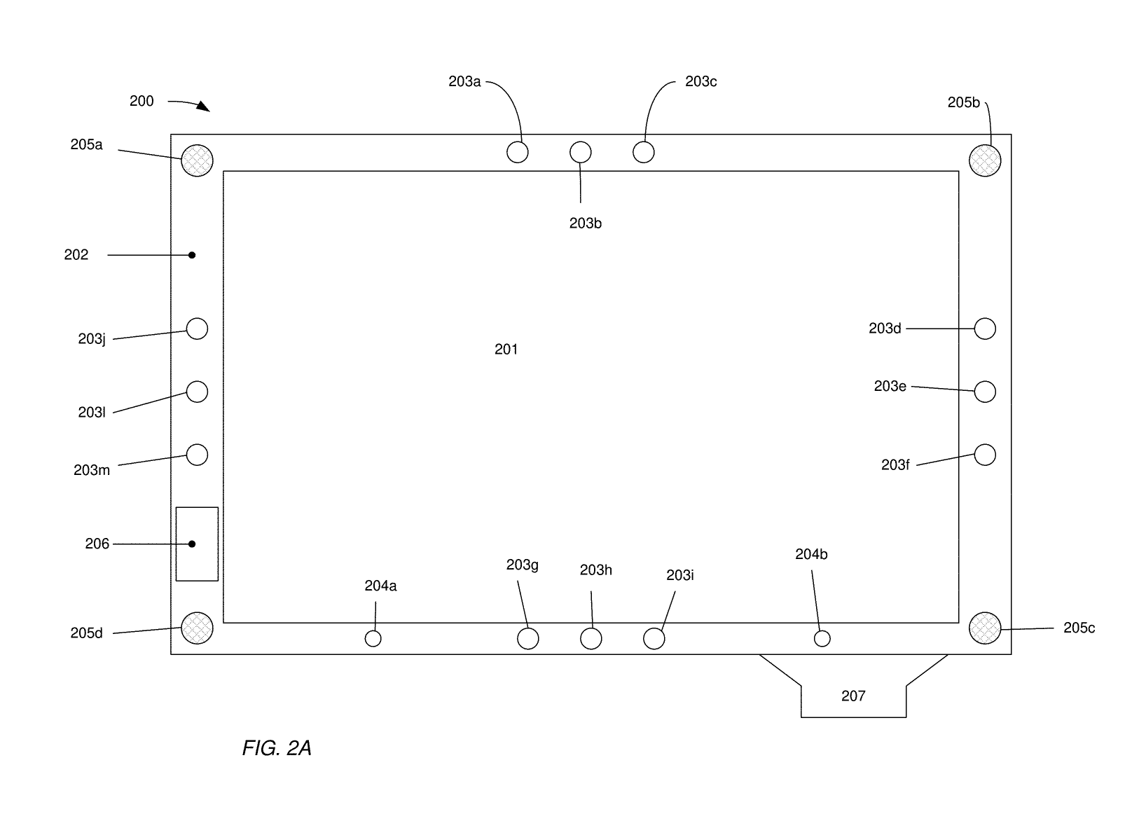

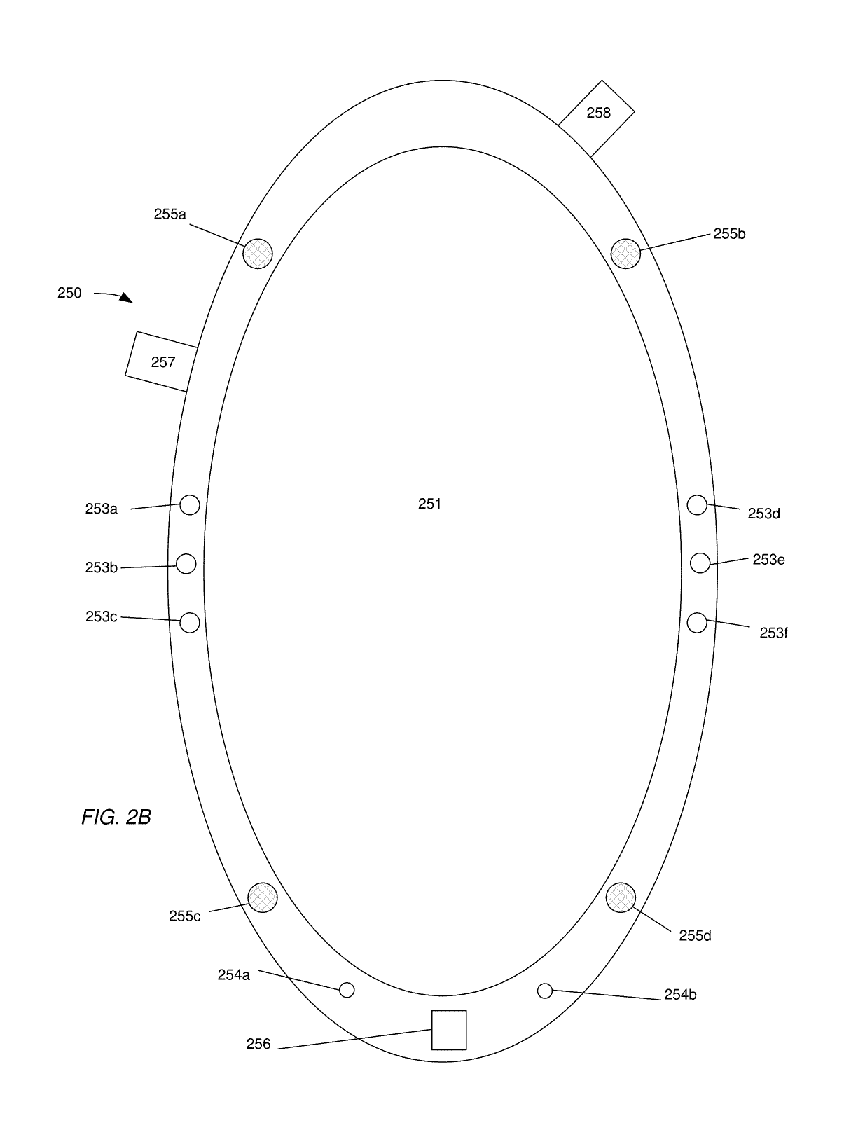

One embodiment of the present design is presented in FIG. 2A. The device 200 includes a central element 201, typically a mirror that operates as a display screen as well. Such devices are available from, for example, Ox-Home (ox-home.com) or Samsung, with the Samsung product called the LED mirror TV. Edge element 202 may be provided and made from any suitable material and shown in FIG. 2A are cameras 203a-1, microphones 204a and 204b, speakers 205a-d, and sensor, voice, or fingerprint reader 206. The sensor may be any type of sensor (biometric, heat, or otherwise), and a voice recognition component may be present, operating separately or using microphones 204a and 204b together with voice recognition software. Also shown in this view is an optional pill dispenser tray 207. While a certain number of components are shown in FIG. 2A, it is to be understood that any number of components may be employed and the invention is not so limited.

The user may stand in front of device 200 and view her reflection. She may verify her identity using fingerprint reader 206, where fingerprint reader 206 may connect to a processor as discussed below and may be able to verify a user's identity. Device 200 may photograph or visually record the user using one or more of cameras 203a-1. The user may speak words that may be received by microphones 204a and 204b, and the device 200 may speak words or provide audio information using speakers 205a-d.

Not shown in FIG. 2A is a series of connection slots in the device, typically in the FIG. 2A embodiment positioned on the outer edges of the device. The connection slots enable the user to connect modules having specialized functionality to the device for the purpose of facilitating personal care. A user can plug one or more modules into the device and can obtain desired functionality at a given point in time, such as a module to determine blood pressure, to determine weight (e.g. a connection to a scale), determine glucose level, and/or to track physical activity, among other functions. One example of connection slots in the device is a USB (Universal Serial Bus) connection, but any type of connectivity may be provided. For example, the device 200 may include a receiver or transceiver while a remote device or module may include a transmitter or transceiver, or the device 200 may include a connection slot for a receiver or transceiver, with connectivity to the device 200 provided via a slot, such as a USB slot.

Device 200 may provide wireless or wired connectivity, including reception of wireless signals or transmission of wireless signals, and may offer any other type of connectivity to a remote computing device as may be known in the art.

FIG. 2B illustrates an alternate embodiment of the device. The device 250 in the FIG. 2B embodiment is oval in shape, again with a central screen 251 that is both a mirror and a display. Edge element 252 may be provided and made from any suitable material, and cameras 253a-f, microphones 254a and 254b, speakers 255a-d, sensor, voice, or fingerprint reader 256 are provided. Also shown in this embodiment are two modules 257 and 258, each providing functionality as described herein. While a generally rectangular and a generally oval shape device is shown in FIGS. 2A and 2B, any form or type of device that provides the functionality described may be provided.

While shown as an integrated device in FIGS. 2A and 2B, it is to be understood that the present design may also include a conversion kit that may employ a plasma or LCD/LED television, including an outer frame that will be able to attach to the central screen and may include cameras, USB outlets and hardware such as a processor, storage, internet connectivity, and/or other pertinent functional components. A program may be provided that allows the user's television to operate in the manner discussed herein. Further, the present device may be incorporated into a piece of furniture, or into a wall, or into any type of other apparatus, either using the full device such as the embodiments shown in FIGS. 2A and 2B or as the aforementioned kit or part of the kit. The device may in various instances be mounted to a wall using mounting hardware known in the art.

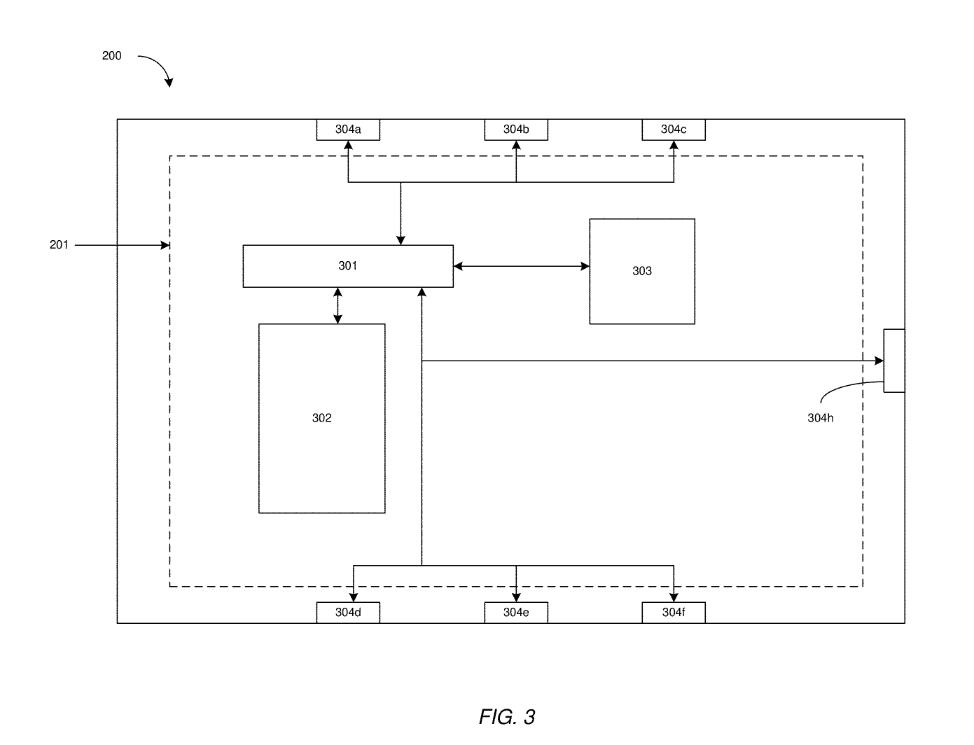

A general view of the electronics employed in the present design is shown in FIG. 3. From FIG. 3, device 200 includes a processor 301 connected to a storage device, such as a memory device 302, and is connected to display 201. The device may include wired or wireless connectivity, shown as element 303. Wireless signals may be transmitted from and received by the device and provided to the processor 301. Connections are provided to multiple connection slots, here shown as connection slots 304a-g. Any number of connection slots may be provided, and these may be USB slots or any type of connection to modules available in the art.

The device, such as device 200 or 250, may be open source in the sense that third parties may manufacture modules or module components that may interface with the device. Standards may be provided such that modules may be produced and desired functionality provided in conjunction with the device and its component parts. Further, the modules may be employed with devices already available, such as a blood pressure module used with an existing blood pressure cuff or other blood pressure evaluation apparatus.

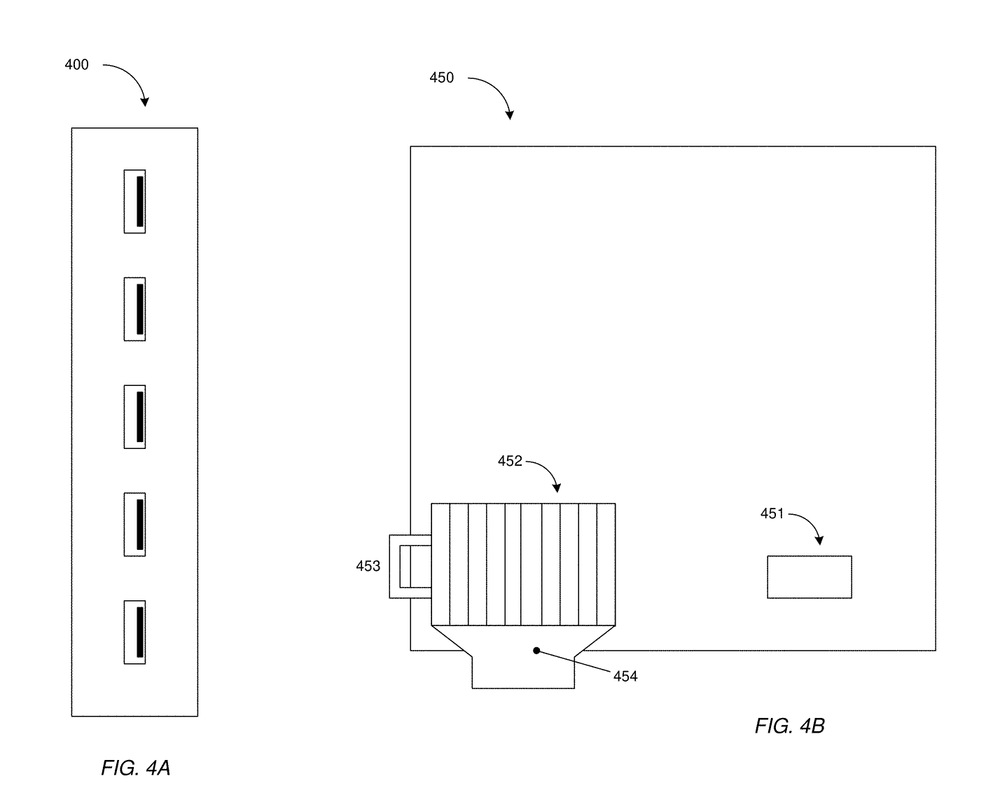

FIG. 4A illustrates a side view of a device similar to that shown in FIG. 2A, where device 400 includes various openings or ports, such as USB ports, 402a-e. FIG. 4B is a rear view of an embodiment of the device 450, including an electrical connector 451 and a pill dispenser 452. The pill dispenser may be a removable feature and may be constructed to accept pills at a given time, with information provided to the processor as to how many and what type pills have been provided, and pills may be commanded to be dispensed by the processor, such as one pill per day if one pill per day is required. A handle 453 is provided such that the owner or some other individual can remove the dispenser or pull the dispenser out for purposes of restocking the dispenser. A tray 454 is provided such that pills or medications dispensed can be provided toward the front of the device and are easily accessible to the user.

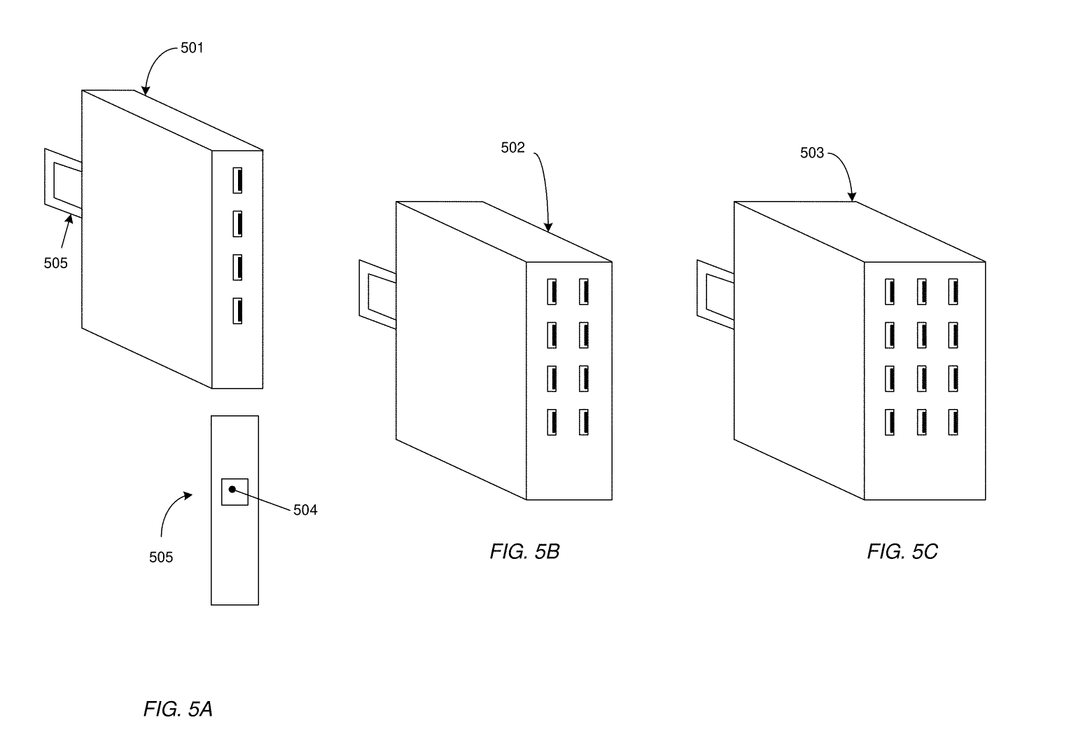

FIGS. 5A-5C illustrate apparatus including a set of separate USB receptacles that may be provided as an alternative to or in addition to the receptacles or slots provided on the device. The apparatus 501 of FIG. 5A, and the other apparatus 502 and 503 shown in FIGS. 5B and C, may be separate from the device and/or may connect to the device via wired or wireless communication. Apparatus 501 of FIG. 5A includes four ports or slots, apparatus 502 of FIG. 5B includes eight ports or slots, while apparatus 503 of FIG. 5C may include twelve ports or slots. Processing functionality may be provided in apparatus 501, 502, or 503, and the user may insert functional modules into the slots provided. Based on the module inserted, data may be collected, or data may be collected and transmitted to the device, such as device 200 in FIG. 2, for processing and/or further processing. As shown in bottom view 505 of FIG. 5A, a slot 504 may be provided for powering apparatus 501. The separate handheld unit described below may include port arrangements similar to those illustrated in FIGS. 5A to 5C, and a handle 505 or other carrying feature may be provided with the apparatus.

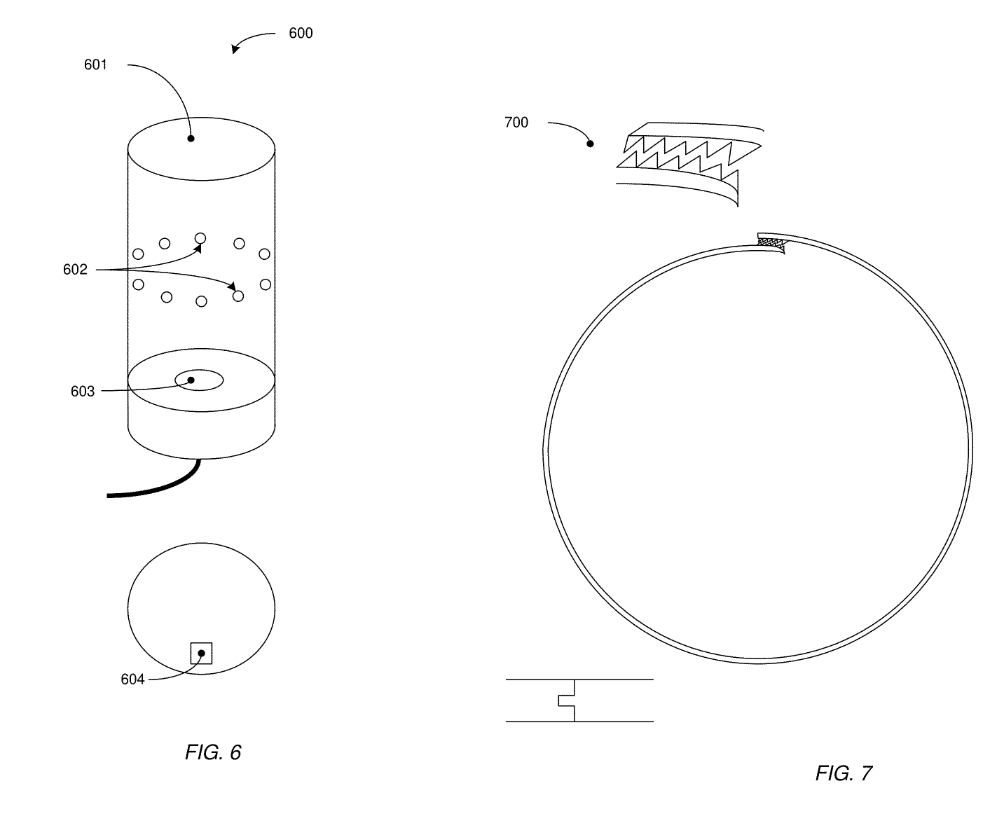

As may be appreciated, any number of functional personal care modules may be provided. As an example, devices to measure pulse rate, blood pressure, body temperature, and so forth may be provided, each with connectivity back to the device, such as device 200. FIG. 6 illustrates an example of a magnification camera 600 that may be provided for any number of uses, including but not limited to examining skin regions, oral examinations, and so forth. Magnification camera 600 includes an open area 601, a set of lights, such as LED (light emitting diode) and/or other lights of different wave lengths (e.g. ultraviolet or black light) employed 602 around the periphery of the interior of the magnification camera, and photographic element 603. The bottom view illustrates a port connection 604, such as a USB connection.

The device may be provided with a module or modules having an ability to read a barcode, including but not limited to a camera such as magnification camera 600. Any type of barcode or QR code or other code known in the art may be employed and may be read. Such scanning functionality enables the device to perform a variety of functions, including but not limited to reading prescription bottles, reading medical products, reading clothing tags and labels, reading cosmetics, reading containers of vitamins, and so forth.

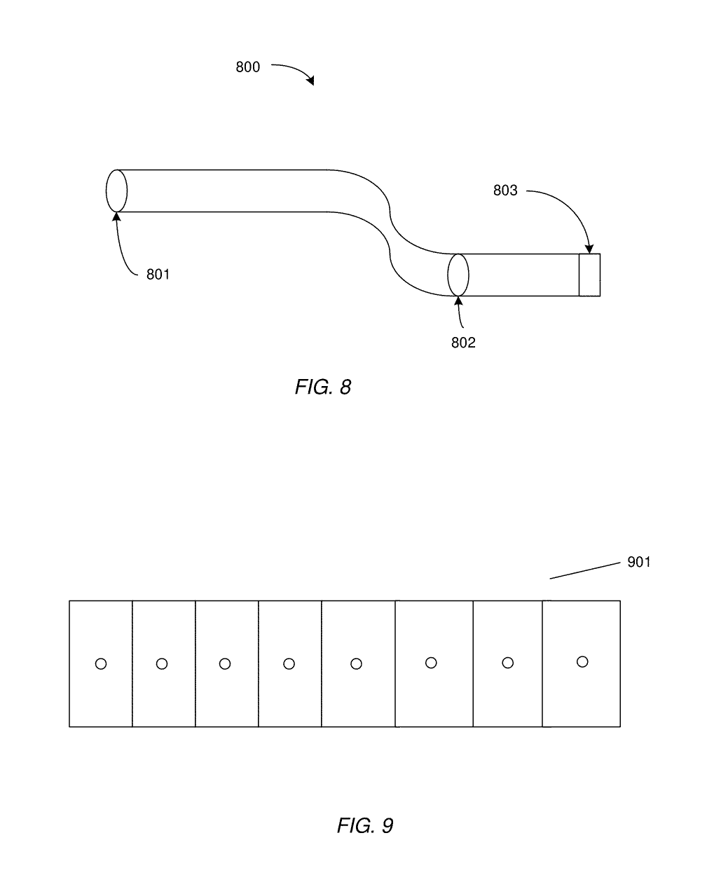

FIG. 7 illustrates an example of a sensing bracelet 700 that may be employed with a module in accordance with the present design. Sensing may be provided for pulse, temperature, and/or other pertinent measurements related to the individual. As an example, the sensing bracelet 700 may include lockable components such as teeth that enable resizing of the sensing bracelet 700. Sensing bracelet may also include a reflective coating or other marker enabling the device to sense the position of the bracelet and hence the user's arm. Such sensing capability may enhance the ability of the device to determine exercise performance and other pertinent information. FIG. 8 illustrates a tape measure 800 including a static reflective bead 801, an adjustable reflective bead 802, and a reflective position stop 803, enabling the measurement of anything, including but not limited to body parts, clothing, hair, separation of feet, and so forth. While FIGS. 7 and 8 do not include connections to modules or the main device, it is understood that such components may either be separate or may be connected by wire, or wirelessly (Bluetooth, etc.) to the device or a module as appropriate. In certain instances, minimal or no connection may be provided, such as no physical connection but an ability for the device, via microphone, camera, etc., to determine position of particular components. In FIG. 8, for example, tape measure 800 may not be connected by wire or wirelessly to a module or the device, but the device may employ a camera to determine positions of the static reflective bead 801, adjustable reflective bead 802, and/or reflective stop point 803 and use the measurement to determine the size of a desired object or objects.

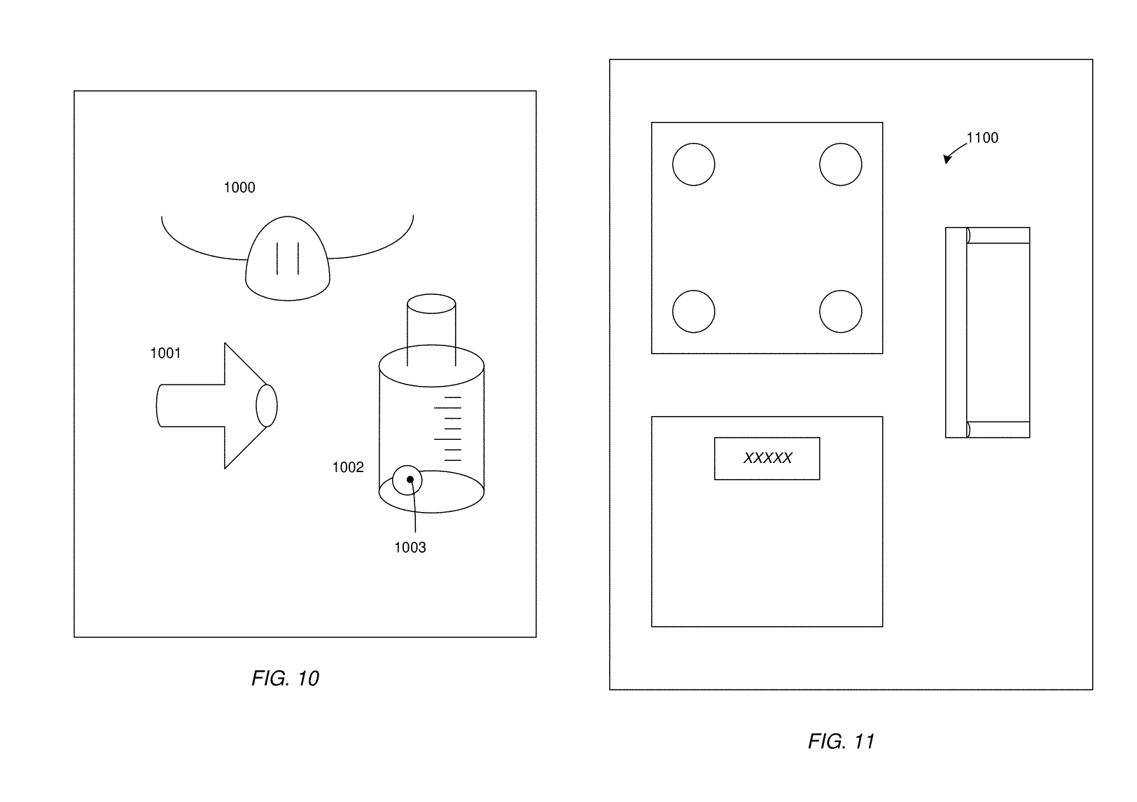

FIG. 9 illustrates a reflective band or strap 901 that may be used to measure head parameters, including temperature, EEG, sweat, and so forth, and may include massage beads. Reflective measuring markers, such as lines or points, may be provided. FIG. 10 illustrates a spirometer 1000, mouthpiece 1001, and a bottle or container 1002 marked with measurement markings, typically reflective, and including a measurement ball 1003, also typically reflective. FIG. 11 illustrates a portable scale 1100, employed to weigh applicable items as well as acting as a turntable where a user can stand in front of the personal care device and the turn table rotates the individual to any desired degree of rotation needed. This can also be used as a base for an inflatable or non-inflatable mannequin of the user for purposes of digitizing garments of clothes, accessories or otherwise implementing physical organ changes on the mannequin prior to desired change on the consumer for example but no limited to the suggested sites of body part enhancements or wrinkles treatment with Botox.

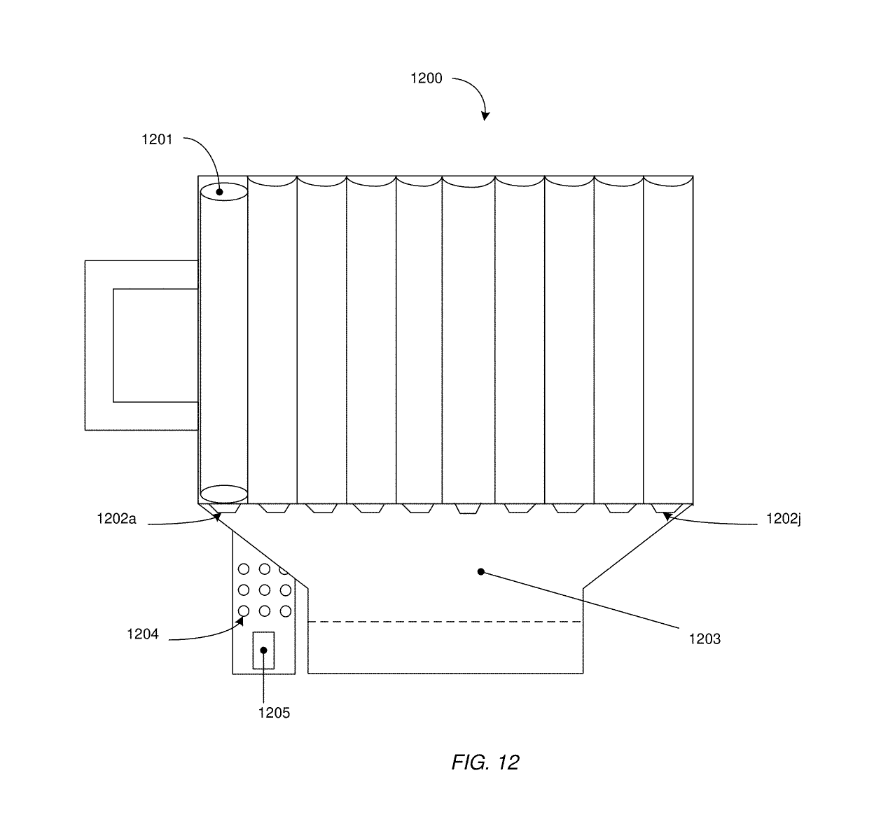

FIG. 12 illustrates a pill dispenser 1200, including a locked compartment 1201 for controlled substances, having programmable dispensing windows 1202a-g configured to control disbursement of pills, a tray or holder 1203, manual pill dispensing buttons 1204, and a fingerprint security reader 1205. The pill dispenser 1201 may be removed from the device and brought to a qualified individual, such as a pharmacist or pharmacist assistant, who can provide pills and/or controlled substances for use by the user. Sufficient security is provided, such as using fingerprint security reader 1205 or other device (sensor, voice recognition, etc.) and manual pill dispensing buttons 1204 and the programmable dispensing windows, where ten such programmable dispensing windows are shown in FIG. 12 and programmable dispensing windows 1202a and 12021 are identified. In this arrangement, the user and only the user or an individual identified as responsible for the user can obtain the necessary pills and/or medications. While shown as a pill dispenser, other forms may be taken to dispense bottles, a door that can be opened and closed with sensing provided, and/or other appropriate containment and disbursement hardware. Pill dispensing may entail the device reading a barcode label of the pills being inserted into pill dispenser 1200, and the device may receive and/or provide pill count, dosage, warnings and related indications.

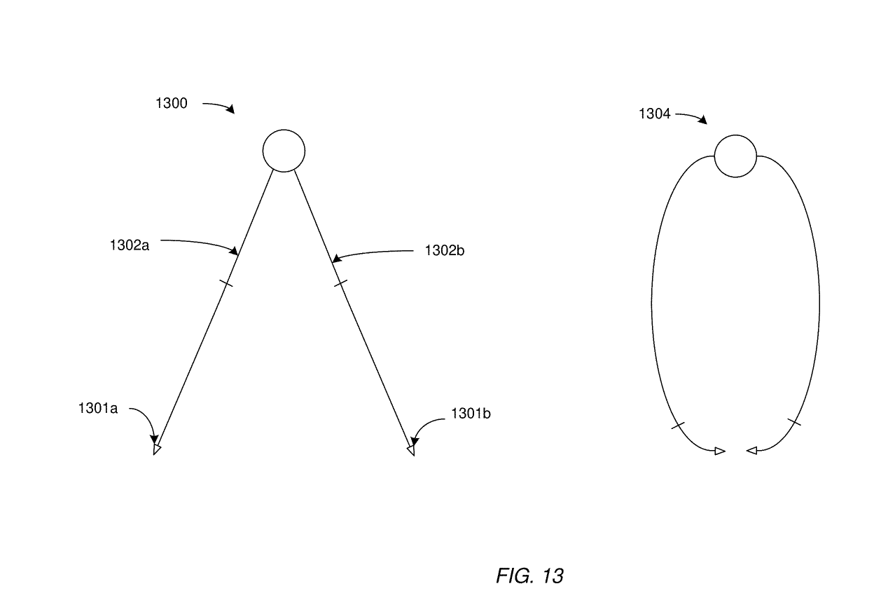

FIG. 13 illustrates a representative caliper 1300, including reflective tips 1301a and 1301b and telescopic arms 1302a and 1302b meeting at jointed element 1303. An alternative caliper is shown as caliper 1304. Reflective points may be provided.

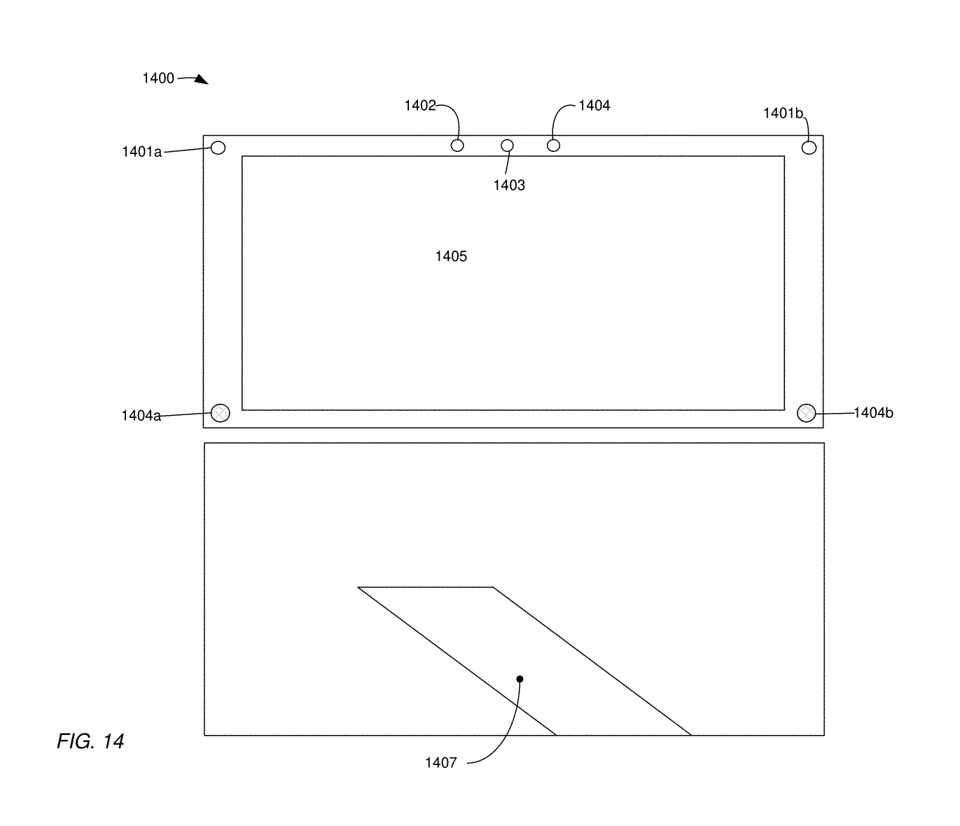

FIG. 14 illustrates a handheld unit 1400. Handheld unit may be carried by the user or other appropriate person for various purposes, including but not limited to collecting data at a remote location, such as a gym, and/or transferring data to a remote location, such as a third party's computing device. Processing functionality is typically offered in the handheld unit 1400, and the handheld unit 1400 typically takes the form of a smartphone or other appropriate handheld device. As shown in FIG. 14, handheld unit 1400 includes a microphone or microphones 1401a and 1401b, a thermal camera 1402, a 3D camera 1403, and a biometric camera 1404, screen 1405 which may or may not be a mirror screen as with the device, and speakers 1406a and 1406b. Screen 1405 may be a touch screen or may be used as a sensor. On the back of the handheld unit 1400 may be provided a stand 1407. A sensor (not shown) similar to the sensor on the device may be provided for authentication purposes (fingerprint, voice, biometric, etc.), or the screen 1405 may be used as a sensor for authentication purposes.

Not shown is the ability of the handheld unit 1400 to connect wirelessly or by wire to either the internet, a cellular service, or other communication service, and the functionality provided may include the ability to collect user data and report the user data to the device and/or obtain user data from the device and/or a remote server or remote arrangement and convey the information to the user or provide the information to a third party. Data may be collected and downloaded at a later time to the device when within range of or communicatively connected to the device, such as device 200. Certain module connectivity may be provided such as via a port or connection.

The handheld unit 1400 may be incorporated into an existing device, such as an automobile part (armrest, headrest, dashboard, console, etc.) or airplane part (armrest, headrest, etc.) or any other type of device. The handheld unit 1400 may be incorporated into devices such as gym bags, purses, clothing (coats, etc.), or even pieces of furniture or other apparatus.

Additionally, but not shown in the foregoing illustrations, a keyboard, either hardware or software, may be provided with the device, such as device 200, or with the handheld unit 1400 such that the user may enter information, request processing, request uploading or downloading of information, and so forth.

Modules provided may include one or more of the following and/or any other combination of devices and/or functionality generally directed to personal care. Modules may include 3D camera-user recognition, recognition of the user using a three dimensional camera or multiple cameras (3D, planar, or holographic), enabling creation of an avatar or virtual avatar of the user, a thermal camera module configured to detect body heat of user, as well as user movements and health related temperatures, a biometric camera module, a scale similar to that shown in FIG. 11 to determine weight, fat, visceral fat, water and muscle content of user, and a heart monitor module configured to determine and provide the heart rate and ECG of the user, track activity of the user as well as changes in heart rate during exercise and/or for the purpose of providing medical alerts. Other modules may include a glucose meter to take blood sugar readings and track blood sugar of the user and may be employed with a medical alert, an oximeter or oximeters that monitors oxygen levels of the user, also possibly used in conjunction with a medical alert, and a blood pressure module that takes blood pressure readings and tracks user blood pressure and may be used with a medical alert. In the present design, connectivity to the device, such as device 200 is provided, and the modules may take any form, such as a small electronic component connected by wire or wirelessly or by any other functional connection to a device, such as in the case of a blood pressure monitor to a blood pressure cuff and inflation device, together with a sensor that senses the blood pressure once the cuff is inflated to the required level.

Medical alert and home alert capabilities may be provided as the device may listen for trigger words as “HELP” or “911.” The device may also provide or be connected to a portable panic button.

Functionality and modules provided may also include retinae scanners enabling the identification of the user as well as monitoring eye health and changes, and a finger print reader or readers usable as a security feature and providing temperate reading and other health screening tests.

Modules may take any form, but as an example, they may be the approximate size of a modern car key module and may have a lithium or other appropriate battery in addition to the USB connectivity for both charging and data transmission purposes.

During operation, the present design may employ the video screen of the device and/or the portable unit to display graphical information, alerts, data collected from the modules and/or related devices and may provide information in the form of reminders or even commercials. The device may employ infrared reflectography using 3D laser scanning to create 3D topographical maps of different body parts (face, muscles, breast, feet, hands, teeth, and so forth) using either reflected infrared or lasers of different strengths. Different readings and different algorithms can be employed for different parts of the body, e.g. bumps and lumps on the skin surface, different angles of inclination, joint range of movement, and so forth.

Modules may be manually or automatically updated the user may choose, via the device, combinations of inputs into the modules. The device may provide face recognition capabilities and may be used to recognize third parties (family members, caregivers, pets) and may potentially be employed to sense intruders. The device may take on the attributes and functionality of a computing and/or communication device and may be placed in a sleep mode and awakened through voice activation or other audible communication, e.g. finger snap, clap, or even a button. The device can employ the cameras provided to track eye movement and/or focus on a certain part of the body, and may enlarge the body part or move it through certain ranges of motion. In the fashion area, a user may try on a piece of clothing and the screen of the device may ask the user to turn around and may show the user how the back of the garment appears. In the cosmetics area, cameras may record multiple versions of the same general skin area when the user employs different cosmetics on the area for the purpose of allowing the user to compare and contrast different cosmetics.

The device typically operates using an open platform that integrates data input and reception in conjunction with applications specifically designed for the device. When using the handheld unit 1400, the user may have available a portable accessory to carry one or more modules, such as a glucometer module if eating or a fitness module (measuring heart rate, movement, etc.) if going to the gym.

In operation, user interaction with the device and/or modules may entail the collection of data that may be retained at the device, with data provided from modules to the device via WiFi, Bluetooth, or through a USB connection or by any other means known in the art. The processor at the device may process the data using device formulas and/or applications and may format the data into graphs, charts, diagrams, virtual assistants and other forms to be displayed to the user via, for example, the device (2D, 3D, or holographic) screen. The data and/or information may be controlled by the user and may be sent from the device, such as device 200, to a remote location, i.e. a virtual “cloud,” where the information may be collected, analyzed, and/or stored. Once transmitted to the remote devices, the user information may be maintained, analyzed, and specific user recommendations or information may be transmitted back to the user. As currently configured, varying levels of service may be provided. As one example, a gold/silver/bronze level of service may be provided, wherein bronze is simply maintaining data at a remote site, silver is analyzing data and providing recommendations, and gold is a concierge type service where the user may be provided contact with available personnel (physicians, pharmacists, personal shoppers, cosmetics specialists, optometrists, dentists, etc.) and particular needs will be addressed. Different or alternate levels of service may be provided.

As may be appreciated, virtually any type of information may be collected and/or provided using the device/module arrangement provided herein. The ability for the user to indicate specific needs and desires via a keyboard and possibly a mouse, and for the device to display information and act as a mirror enables a virtually unlimited range of functions. A particular user may wish to receive clothing recommendations and may have her skin tone and hair color determined, and clothing color recommendations provided. Body type may be determined, and age entered, and age appropriate wardrobe selections may be provided based on an analysis provided from the remote location. Another user may have a blood sugar issue, and his blood sugar may be monitored and tracked, and based on his history and desires, recommendations as to what to eat and/or when to eat may be provided to the device and/or to the handheld unit 1400. Another user may want to monitor stock quotes and baseball scores in addition to tracking his progress on a weight loss program. While providing stock quotes and baseball scores is not strictly a personal care function, the present design may offer any type of functionality offered by a computing device and/or tablet and/or smartphone. Thus the user may be exercising with the handheld unit 1400 tracking his progress, i.e. time spent exercising on a treadmill, while the device may display stock quotes or baseball scores. At the end of the user's workout, he may weigh himself using the scale module and may receive meal recommendations and/or a graph of his exercise progress or weight via the device. Thus single or multiple functions and determinations may be provided or made based on functions, applications, and computations available, as well as the needs and desires of the user, in addition to the functionality provided at the remote device arrangement.

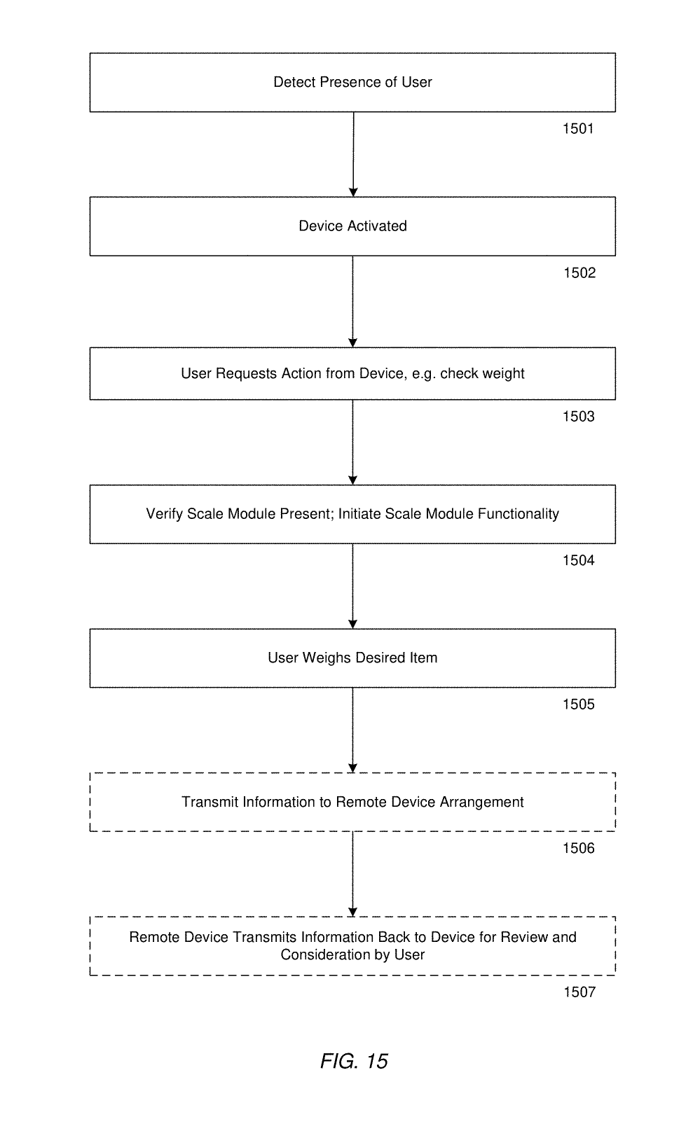

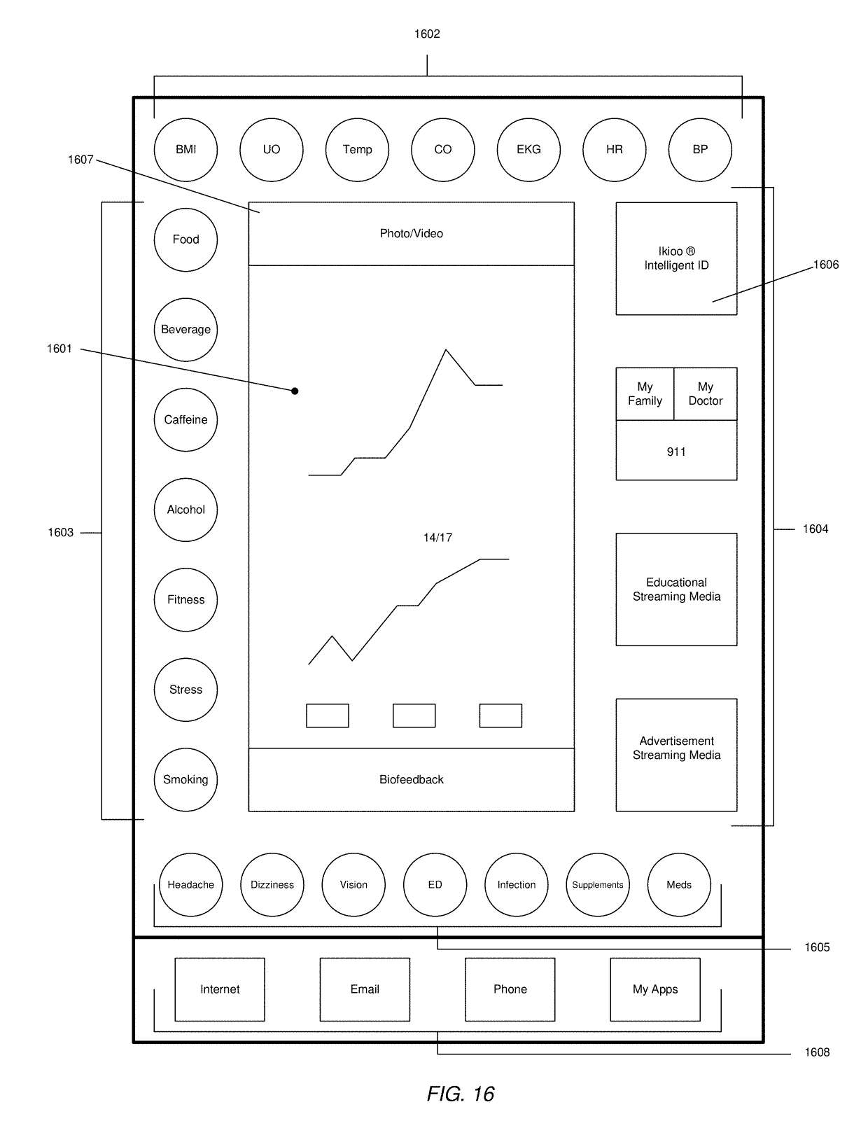

A simple functional example of use of a scale module is presented in FIG. 15. From FIG. 15, the device, and more specifically the cameras on the device or the fingerprint reader or other device component, detects presence of the user (e.g. pressing a key on a connected keyboard) at point 1501. At point 1502, the device, such as device 200, is activated and may come out of a sleep or hibernation state as appropriate. At point 1503, the user may ask for an action from the device, in this case checking the weight of a desired item using a scale. The device determines whether the appropriate module is connected or available, here the portable scale module. If not, the device prompts the user to connect the module to the device, and the scale module functionality is initiated as shown in point 1504. Weight information may be provided on the screen of the device, and specific information may be provided—commercials, recommendations, graphs of weight readings over time, most recent weight, and/or other information.

At point 1505, the user weighs the desired item, and the resultant weight is provided to the device, and more specifically the processor in the device. The screen of the device may display current weight and tracked weight, either graphically or numerically. At point 1506, which is optional, the device, such as device 200, may transmit the information to a remote device arrangement for further processing, such as determining recommendations for a man the user's age and in the user's situation. At point 1507, the remote device arrangement may send user specific information back to the device, such as device 200, for review and consideration by the user. Emails, text messages, phone messages, and/or other user selected communication methods may be employed to inform the user of determinations made at the remote device arrangement. Certain information may be provided from the remote device arrangement to the device to enhance operation, such as locally storing recommendations for the particular user determined at the remote device arrangement. The device may at that point return to a hibernate or sleep mode or may continue operation as desired.

Functions may vary and may depend on what is required, and as may be appreciated, any list of functions may be changed, improved, reduced, or otherwise altered depending on a variety of factors, including but not limited to popularity of the functions, needs and desires expressed by users, use patterns, costs associated with apps and functions, and so forth. Certain functions may be provided either locally, i.e. at the device, or remotely, i.e. at the remote device arrangement, or a combination of both. In general, the system, including the device and remote device arrangement, receive information and queries and respond appropriately for personal care issues that arise.

Examples of the determinations made and functions provided are as follows. In the area of health, lung health may be maintained and monitored, and the device may determine respiratory rate (RR), CO₂ level, lung volume, pulse oximetry, breathing characteristics, jugular vein distension (JVD), pitting edema, and/or body pH level. The device may respond with a chart including a degree of hypoxia, a graph with the user's lung volume as compared to users of similar age and sex, and a pitting edema score. In the area of heart health, the device may collect pulse/heart rate, blood pressure, heart rhythm, and EKG values with appropriate modules. The device may determine and/or report a graph, raw data, and or percentile data, as well as recommendations. In the area of nose and sinus health, the system, including the device, handheld unit, appropriate modules, and/or remote device arrangement, may analyze the nose for deviations in shape and/or color, flaring, discharge, and nasal patency. The system may then provide raw data, a digital model of the nose and/or sinus, and provide a percentile, score, and/or chart. In the field of eye health, the system may assess or determine sclera color, visual acuity, peripheral vision, analyze the pupil, iris, sclera, and so forth, determine eye pressure, redness or dryness, and eyelash evenness, and the system may provide a digital model of the eye, a scoring of quantities such as peripheral vision, raw data, percentile values, a chart, and a chart or graph.

Many, if not all, of the health related evaluations may result in a combination of a score or scores, raw data, percentile, and a chart or graph of the health of the particular site. It is to be understood that these and other relevant information (recommendations, warnings, pictures, etc.) may be determined and provided as appropriate, but that all such determinations may be made and provided when assessing health parameters.

In the area of ear health, the system, again including the device, handheld unit, appropriate modules, and/or remote device arrangement, may assess the internal ear, including the external, middle, and/or inner ear, analyze the external ear (auricle and pinna), determine degree of hearing loss, and perform a bone conduction evaluation (Weber/Rinne). Dental or oral care health may include an assessment of lips, inner and buccal mucosa, tongue, mouth floor, teeth and gums, hard and soft palate, salivary glands, and tonsils. Many of the assessments discussed may be made using a camera or recording device, but others may require modules specially made for the task. Digestive health may be assessed using food intake, stool analysis, BMI (body mass index), and weight. Liver health may be assessed using skin color, sclera color, ammonia levels in urine, level of consciousness, abdominal distension, weight, total body water, skin irritation, activity level, and/or stool color. General hormone balance may be assessed according to a hormonal profile, activity level, weight gain, EKG, heart rate, mood, level of anxiety, headaches, and/or sleep pattern. Kidney health may be assessed according to daily urine output, blood pressure, glucose level, and/or degree of edema. Muscle health may be assessed according to strength, tone, size, symmetry, fasciculations, tremors, arm/leg circumference, and activity tolerance.

In the present design, the user may be monitored using a module or the user may be queried directly or may offer the information requested. For example, the user may be queried as to “have you experienced any muscular tremors in the last week” and she may respond “no.” Alternately, the user may be asked “what has been your activity level in the last day?” and may provide the user with options (none, mild, moderate, extensive, or quantities, such as ran for 20 minutes). Various options regarding obtaining the necessary information, including the necessary health information, may be provided. The user does not need to provide the information—the information may be provided by a third party (caregiver, spouse, etc.)

Nail health may also be assessed, analyzing convex curvature, i.e. the angle between the nail and the nail bed, and in addition to the other reports and/or information provided to the user, he may receive a graph of a degree of clubbing (normal, early, or late), based on nail bed shape and angle. Testicular health may be assessed according to a pain scale, new lumps, heaviness in scrotum region, and/or testicular swelling. Obstetric health maybe assessed—weight, heart rate, blood pressure, portable ultrasound results, fetal heart rate assessment and analysis, and food intake, for example. Bone health may be assessed according to bone density, calcium level, activity level, pain scale, swelling, nutritional intake, and a bone densitometer score. Mental health may be determined by assessing speech recognition, level of consciousness (LOC), weakness on one side, blood pressure, visual changes, and/or NIH score. Ovulation health may be assessed by determining a surge in Luteinizing Hormone (LH) urine, analyzing cervical mucus consistency and/or basal body temperature (BBT). In addition to the other health information provided (raw data, graphs, charts, etc., ovulation results may include a digital calendar or listing of best ovulation times.

Spine health may be assessed according to structures or deformities, warmth, swelling, range of motion (ROM), presence of scoliosis, and gait. Joint health may be assessed by evaluating warmth to the area, ROM, swelling, presence of nodules, and/or gait. Skin health may be assessed according to skin turgor, color, skin breakdown, and degree of edema. Hair health may be assessed according to hair distribution, hair density, color, moisture content, dryness, texture, and presence of infection and/or infestation. Breast health may be determined based on swelling around the breast and armpit, pain level, changes to nipple, discharge, signs of lumps, and/or menstrual cycle issues. Information provided relative to breast health may additionally include a video showing a proper breast exam, and a digital model of the breast of the user or a hypothetical breast, again for examination purposes.

Foot health may be determined by collecting signs of infection, color, hair distribution, skin integrity, and degree of pitting or edema. In addition to the other reporting data, the system may provide a digital model of feet or the lower extremities. A general collection of parameters for the user may occur, including but not limited to collecting heart rate (HR), temperature (T), basal body temperature (BBT), respiratory rate (RR), SaO2, pulse oximetry, CO2, systolic blood pressure (SBP), diastolic blood pressure (DBP), urine chemistry, dry blood work, joint ROM, spine flexibility, muscle strength, BMI, total body water (as a percentage of body weight) in relation to age and sex, electrocardiogram (EKG), lung capacity (TV, TLC, VC, FRC, RV), height and weight, and body pH level. These and other pertinent values may be collected and reported and/or continuously tracked.

In the area of fitness and wellness, different module specific interactions may occur where data is received by the system (device, handheld unit, remote device arrangement, module(s)) and certain information assessed and provided. In the area of fitness, the system may collect or receive user goals, weight loss values (current or desired), general fitness, preparation for an athletic event, muscle building, and rehabilitation from injury. Output may include an individualized fitness program as well as graphs and charts and recommendations. In the area of body building, height, weight, neck, chest, upper arm, waist, hips, and any other relevant area may be measured, and information about body fat provided as well as changes in body measurements. For sweat, the device may be provided with a module that analyzes user sweat and provides information on total free amino acids, ammonia, protein concentrations, and nitrogen balance of the user, detects a negative nitrogen balance, gives dietary recommendations, recommends post workout recovery meals, and recommends a dietary and supplement plan. For workout performance, the system, and possibly a module in the system, analyzes body mechanics during exercise by measuring weight distribution, relative angles at joints during exercise or body heat via thermal infrared camera. The device provides corrective user feedback on proper body mechanics during exercise. In the area of nutrition, the user may input daily food intake via picture, barcode scanner, or may select items consumed from a pull-down menu. Also provided or calculated are total calories, protein, carbohydrates, fat, and fiber. The system may recommend dietary changes toward fitness goals and may provide charts and graphs.

In the area of exercise, the system may collect or receive user submitted photos of gym equipment (at home or in gym), may enable interaction with an Al Personal Trainer, where the user communicates in real time with the Al Personal Trainer, time between sets, number of reps and weight lifted, body posture via angle of shoulders to waist and feet, and the system may recognize available equipment and build an exercise program. A trainer may observe the user while completing exercises and give real time feedback about technique. The system may provide motivational instruction. Recommendations by the Al Personal Trainer may optimize user exercise output. Physical health at work may be assessed by calculating angle of the body while seated and doing work, assuming the device or handheld unit is available at a representative workplace. The system may also calculate height of desk, chair, keyboard, and mouse. The system may recommend corrective body posture and ergonomic corrections to the work station. Physical health in the case of injury may be assessed, wherein a provider or the user or other appropriate individual may input the user's injury or injuries. The device may then create an exercise regimen based on the user's needs and specific injury.

In the wellness area of sleep architecture, the system may measure room temperature, amount of room light, noise, track user sleep/wake cycle, and the amount of movement during sleep. The system may recommend changes to sleep architecture. Tracks changes and saves users optimum sleep requirements. The system may also address menses, determining date of first day of the menstrual cycle, length of bleeding, bleeding volume, daily temperature, and may provide recommended dietary changes.

With respect to fashion, the system may assess body shape measurements, i.e. biometric points, body shape, and may measure four separate points—shoulders and hips, in the form of an “X.” The system, including but not limited to the remote device arrangement, may provide recommendations on clothing style and cut, perform “body blocking,” and provide a “virtual 3D mannequin” of the user based on calculations. The user may be allowed to “try on” various styles/cuts/colors of clothing on the virtual mannequin. Based on mannequin sizes, retailers will have better information on the user's particular clothing needs and wants. The system may also assess secondary body measurements, determining height, weight, BMI, arm length, arm circumference, thigh circumference, torso length (long, short, average), breast size, shoe size/width. From these secondary body measurements, the system may determine detailed body composition, make specific suggestions as to clothing, styles, and accessories through the device and available from retailers, and creation of a “virtual mannequin” can increase details known about the user's body image to both the user and a retailer. The system may also determine body size changes, assessing minor and major changes in weight and body changes due to pregnancy, and providing revisions or potential revisions to style, size, color, and cut of clothing, as well as suggestions.

The system may also assess user demographic information, such as age, race, geographic location, profession, income, education, marital status, and number of children. From this, the system can determine a presentation specifically tailored to the user, determine style suggestions, price points, and clothing styles relating to seasons, temperatures, and precipitation, and allows retailers to focus on a specific consumer profile. The system may additionally determine clothing wear, possibly employing a sensor in user clothing, to determine date of wear, number of wearings, and frequency of wearings.

With respect to cosmetics, the system may assess tone and pigmentation by determining skin undertone based on the color of the major vein around the wrist and by blue, yellow, red content against a white background. Results determined may include a 3D graph, a personal color wheel for hair, a personal color wheel for make-up, prescription skin care recommendations, over-the-counter skin care recommendations, peel recommendation, laser resurfacing options, and continuous updates and recommendations. Skin category may be assessed, including determining consumer inputs, oil content of skin, and reflexive skin properties, and providing a graph of skin, oily to dry, recommended moisturizing and cleansing creams. The system may also evaluate pores, determining distribution and spread measured by number per square centimeter and measure color and size of pores. From this information, the system may provide information including a graph, chart, updates and recommendations, microdermabrasion, chemical peels, facial cleansers, and creams.

Acne may be assessed according to distribution and spread measured by number per square centimeter, measurement of color and size of pores, acne location via a topographical map of the user's face, depth and height, and cystic versus black head. Information provided and reported may include a graph, chart, topography, updates and recommendations, microdermabrasion, chemical peels, facial cleansers, creams, laser resurfacing, and information regarding or from a dermatologist. Scars may be assessed based on discoloration, size, location, depth, and height. Again, based on this, the system may recommend or provide a graph, chart, topography, updates and recommendations, microdermabrasion, chemical peels, facial cleansers, creams, laser resurfacing, and information regarding or from a dermatologist. Skin elasticity may be assessed based on skin turgor, muscle and skin laxity, and nasolabial fold measurement. The user may be provided with a graph, topography, charts, updates and recommendations related to skin elasticity, firming products, and/or laser tightening methods.

Wrinkles may be assessed based on measurements of crow's feet around the eye, length, depth and quantity of wrinkles in particular regions, frown lines, forehead wrinkles, wrinkles around the mouth, wherein all wrinkles may be assessed based on wrinkles per square inch. The system may determine and provide a graph, chart, topography, updates and recommendations, cosmetic grade skin care, medical grade skin care, Botox treatments, fillers, chemical peels, and/or specialized laser procedures. As may be appreciated, more severe skin issues may call for added recommendations, procedures, products, and/or treatments.

Facial hair may also be addressed by determining follicles per square inch, coarseness, length, diameter, color, and/or location, and the system may provide graphs, charts, updates, and recommendations relating to waxing, medicated creams, and laser hair removal. Facial attributes may be assessed and recommendations or information provided. Facial attributes evaluated include shape, position of eyebrows relative to eyes, distance from medial eyebrow to medial canthus, distance from lateral eyebrow to lateral canthus, distance of lateral canthus to medial canthus, spread and distribution of hair per square millimeter, length and diameter of hair, shape and contour of eyes, diameter of iris, distance measurements of eyebrows, ridge of nose, and forehead/hairline, width of eyelids, distance of the upper eyelid from the lower lid at multiple locations, eye color-measure by the three basic color red, blue, yellow, color and discolorations of sclera, eyelashes measured by follicles per square millimeter, coarseness measured by the length, and diameter of each hair, with information provided including graphs, charts, topography, updates, and recommendations regarding shaping, make up, medications, Botox, and even surgery.

Facial symmetry of hair may be assessed, determining length of forehead and measurement of trichion to glabella. The system may recommend facial hair profiles and may provide graphs, charts, and updates. General face shape may be assessed, including measurements of trichion to menton, ear lobe to ear lobe, trichion to glabella, trichion to arch of eyebrows, infratip of nose to menton, menton to mandible, mandible to pre auricular points, and/or length of filtrum. Results provided may include graphs, charts, topography (as used herein, topography includes maps and or data related to topography of the particular feature(s) or region), updates, and recommendations including hair, make up, and jewelry.

Measurements and determinations of particular features may be made, including but not limited to nose, lips, cheeks, jaw lines, chin, and neck, with assessments made using cameras and any other appropriate devices or modules. Graphs, charts, topographies, and updates may be provided as well as feature specific recommendations, such as makeup, surgical procedures, makeup, fillers, moisturizers, treatments (e.g. Botox), and so forth.

Teeth may also be assessed based on measurement of teeth size, separation distance of teeth, angle of teeth, color or shade, and/or color of gums (measured by red, blue, and yellow against white). In this cosmetics area, other outside factors may be assessed—current medications and/or current diagnoses, for example. The system may make specialized recommendations pertaining to skin health with these medications and diagnoses, and may make recommendations of supplements and/or foods to maintain skin health.

Certain personality factors may be determined, such as desired look during the day or night, age, gender, geographical location, income, and/or occupation. From this, the system may determine graphs and charts, and may make recommendations such as color pallets and application instructions.

In the area of pharmaceuticals, the present system may make additional assessments and may determine recommendations and provide information to assist the user in his or her personal care. One area is prescription medications, wherein the system collects user name, quantity, dose, administration, duration of usage, refills, adverse reactions, use in specific populations, over dosage symptoms, over dosage signs, prescriber name, prescriber address, prescriber phone number, pharmacy name, pharmacy address, pharmacy phone number, controlled substance schedule (if applicable), price per unit, and medication expiration date. Certain information may be collected from third party sources if available, as is the case with other functions performed by the system. For example, if the medication is known, the system may submit a query seeking adverse reactions, or if a pharmacist is known, the system may seek the address and telephone number of the pharmacy from an online source. As may be appreciated, such specialized functionality must be provided in the system, i.e. seeking information from a particular site, seeking the specific information needed, and obtaining the information. The device may perform this functionality, or such functionality may be performed by the remote device arrangement or a person associated with the remote device arrangement.

Information provided by the system may include a drug interactions alert, a refill alert, alternative medications options, generic medication options, an electronic update of current medication list to the device cloud, filling of the pill dispenser, lock filling into medication drawers if applicable, providing alternative price per unit information from other suppliers, an expiration date alert, and a next dose alert through cloud based system.

Over the counter supplements and supplies may also be assessed and information provided. Assessments may include name, quantity, dose, administration, duration of usage, refills, adverse reactions, use in specific populations, over dosage symptoms, over dosage signs, prescriber name, prescriber address, prescriber phone number, store name, store address, store phone number, price per unit, and/or expiration date. The information provided to the user may include a supplement interactions alert, a refill alert, alternative supplement options, an electronic update of current medication list, filling of a dispenser, alternative price per unit from other suppliers, and/or coupon options from suppliers.

Health markers may also be assessed. Information assessed may include Indication(s) for particular drugs (allergies, etc.), heart rate, temperature, respiratory rate, pulse oximetry, systolic blood pressure, diastolic blood pressure, urine analysis, blood analysis, breath analysis, weight, body pH level, sclera color, pupil size, lip color analysis, skin color analysis, mini mental status exam, pain scale, electrocardiogram, and/or BMI. The system may take these inputs and make relevant determinations including a score, raw data, percentile, chart or graph, measurement of physical or mental response to a medication or supplement, possible medication or supplement overdose symptoms alert, and possible medication or supplement overdose signs alert.

Dispensing of prescriptions may be assessed and information provided. Assessments may include name, quantity, dose, administration, duration of usage, and refills. The system may provide a visual display of one or more of the following: substance name, substance remaining quantity, substance dosage, route of administration, allowed refills, and/or substance expiration date.

The present design may be used in various scenarios, including but not limited to use of the design for pets or animals as well as plants in addition to human personal care. In the pet scenario, the device may monitor a pet, such as a body temperature of a pet, using a module, and/or the location of a pet, such as using a collar with a GPS component or other position determining device. In the horticulture arrangement, for example, plant health may be monitored and plant care provided as appropriate. In one instance, a device may measure amount of water available to a tree, and if deficient, may notify a person or service to provide water to the plant, or may cause a sprinkler or watering apparatus to turn on automatically.

The system may also employ a health band and other functionality similar to and in the manner of the disclosure of U.S. patent application Ser. No. 14/952,496, filed Nov. 25, 2015, inventor Ayman Salem, entitled “Health Band Apparatus,” the entirety of which is incorporated herein by reference.

### Interactive Diary

The present design further includes an interactive diary function wherein the user can keep track of information and data related to his or her personal care and such information can be employed to better provide personal care for the user, either from an off site location/central server device or arrangement or from the device itself. The interactive diary tracks pertinent parameters to the individual user in the area of personal care and wellness and may offer at least one recommendation. Interactive diaries for personal health and wellness may be offered in but not limited to the areas of health, fitness and sports, fashion, cosmetics, education, travel, finance, nutrition, pharmaceuticals, pets and/or horticulture. For example, the current design may monitor blood pressure and may track blood pressure. However, blood pressure is merely one data point in a plurality of data points related to an individual user, and when blood pressure is considered in connection with factors such as blood pressure readings over time, family history, weight, salt intake over time, smoking history, coffee intake over time, level of exercise, a more complete representation of the person may be determined. In such a situation, the individual may be encouraged to reduce coffee intake and/or exercise in a manner conducive to her ability. For example, if she has back issues, this may be known and encouragement to swim a certain amount per day three days per week might be recommended. A different person with a different profile but an identical blood pressure reading may be encouraged to decrease salt intake, reduce smoking, and to increase exercise by running two miles instead of 1.5 miles every day. The interactive diary function encourages the user to provide more information and obtains a more complete profile of the individual. In another example, the user may seek to purchase a new shirt; certain options may be offered to him, such as collar style, buttons, cut, sleeve length, etc., and the program may recognize prior preferences using the interactive diary and offer certain options to the user based on past history or indicated learned preferences.

A further example is in the area of sports where an interactive diary for a child playing any sport can be functionally tailored to act as an interactive diary used by a professional athlete. The interactive diary may, as one example, collect and track sizes and functionality of certain muscles and ligaments, nutritional requirements and goals, track progression of skill sets desired, as well as vital signs affected by each workout. Also, the desired and available fashions for that sport as well as skilled coaching recommendations tailored to that specific athlete, whether a child or a professional athlete sharing the same sport tenure, may be assessed and recommendations provided as desired.

The interactive diary consists of collecting information at the device in accordance with the foregoing design, including collecting information using the modules provided that are interchangeable and as a result specific to the user. The information collected may be passed to the central server arrangement or an offsite location and may be stored and/or processed offsite. Alternately, the device may store and process the information, or both local and remote devices may offer part or all of certain interactive diary functions.

FIG. 16 illustrates an appearance of a screen operating in accordance with the interactive diary function, and this may be offered on the personal care device discussed above or other appropriate hardware. From FIG. 16, there is provided a central region 1601 and various peripheral regions including upper region 1602, left region 1603, right region 1604, and lower region 1605. A single control button 1606 causes a return to a central or top screen when pushed irrespective of the active screen.

Central region 1601 may include traces of information being measured, i.e. the biofeedback being measured using a module or modules and the mirror device. The buttons presented in FIG. 16 may either be hard, physical buttons or may be soft buttons activated by user touch. Buttons in the top row generally correspond to readings taken of the user's health, including but not limited to blood pressure, heart rate, EKG, cardiac output, body temperature, urine output, and BMI (body mass index). In one embodiment, pushing the button once results in the system showing a tracing of the parameter in the central screen. Pushing twice maximizes the central screen to cover the entire screen, and pushing the button for a predetermined amount of time, such as three seconds, the central screen is maximized and user editing is enabled, such as editing of urine output, etc., using a keyboard, voice, or in some other manner in accordance with the device and system described above.

Left region 1603 includes certain activities that are or may be relevant to the individual user, including but not limited to food consumed, beverage consumed, caffeine consumed, alcohol consumed, fitness and exercise accomplished, stress factors, and smoking. The user may enter what he or she consumed or did, and this may be tracked or attributes displayed. The lower region 1605 may include various symptoms and/or prescriptions taken, including but not limited to choices such as headache, dizziness, vision issues, ED (erectile dysfunction), infection, cramps, and supplements, vitamins, medicines, etc. In this embodiment, a single push of the associated button may provide a time and date stamp and propagates the selection to desired locations, local and remote. Two pushes maximizes the screen and a three second button push in one embodiment allows editing of the entry, such as supplement taken, type of vision issue, duration and severity of headache, and so forth.

A lowest region 1608 includes other relevant functionality, and may include selections for Internet, email, phone, or desired applications or clusters of applications. Different functions may be provided, and selecting these buttons takes the user to the selected option.

A photo/video button 1607 may also be provided. Pushing beverage and pushing photo/video button 1607 causes a photo or video file to be created or identified and the photo or video can be provided to a beverage file storage device or area. In various instances, selection of one of the buttons in left region 1603 provides an associated screen for entry of further information, e.g. quantity of beverage consumed, type of medication taken, headache severity, etc. Selection of the parameters offered, or entry of data according to the parameters offered may be stored and processed locally and/or remotely.

Right region 1604 in the embodiment shown includes four functions. The top button is an intelligent ID (Interactive Diary) button 1606 that returns to a top level for all screens except the main screen. The second button is favorite contacts, and may include name, phone number(s), email address, and may include emergency numbers. A limited number of contacts may be provided, or if desired, as many as requested, or all. Selecting one of the contacts may put the user in touch with that contact. The bottom two elements may be streaming media and advertising if desired by the user and/or a provider, such as a service provider.

The central window, or central region 1601, shows a corresponding page when one of the peripheral buttons is selected. Buttons may change to accommodate enhanced functionality. The corresponding page may be a home page, a selected page, an image, or other appropriate page. The device camera may be activated by selecting “photo/video” on the central region 1601. Selection of “biofeedback” when the user is being monitored provides the monitored results to the user, such as a graph, with other factors provided as appropriate, such as consumption of foods, beverages, smoking, etc. when monitoring blood pressure, temperature, etc. Positive factors may be provided in one color while negative factors may be provided in a different color. Selecting a button twice, such as the biofeedback button, may maximize the biofeedback screen. Selecting the button for a particular amount of time allows the user to edit parameters.

The system may also provide recommendations. Recommendations may be dynamically updated and may include recommendations on food, diet, supplements, hydration, fitness and exercise, biofeedback recommendations including options such as meditation, medication recommendations, including when to monitor intake, sodium intake, alcohol intake, caffeine intake, smoking, and stress avoidance recommendations.

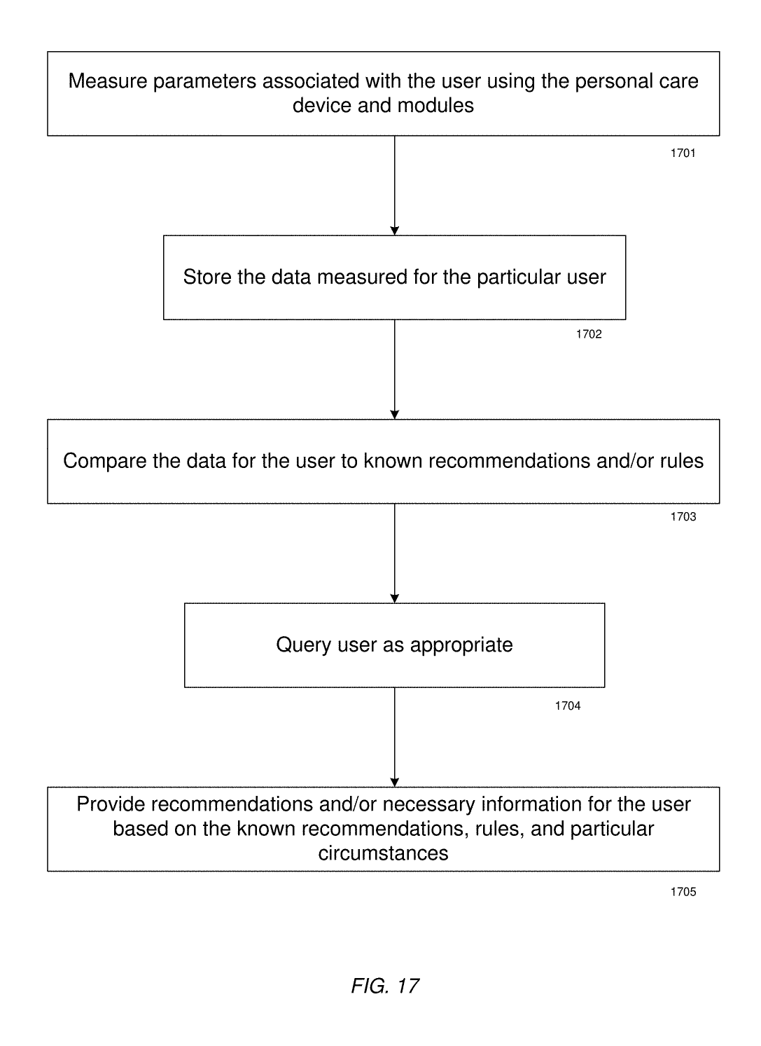

The system may operate according to the representation of FIG. 17 with respect to the interactive diary. From FIG. 17, the device initially measures parameters associated with the user using the device described herein and the various modules applicable to the particular user. As an example, the user may measure her temperature over a series of sessions, or even over a single session. The data measured is stored at point 1702 for the particular user. At point 1703, which may be user initiated, system initiated, periodic, or otherwise instantiated, the system takes available information as appropriate and compares this information with known recommendations and/or rules and may optionally query the user at point 1704 and provides recommendations and/or necessary information for the user in point 1705. As an example of the querying at point 1704, if the patient is a young man who has seen a rise in his temperature, such a rise in temperature may be unexpected but the user may be provided with questions or recommendations such as “are you feeling ill?” “do you have a headache?” “do you have a normal appetite?” and so forth in an effort to identify the problem. The user may be presented with information at point 1705 such as “Jeff, you have an elevated temperature. You should drink fluids over the next 24 hours, and it is recommended you drink six glasses of water in the next day. Please take your temperature again in approximately four hours” or something similar.

While health has been the focus in many of the examples provided, other personal care functions may be similarly offered and buttons provided or added that provide functionality in the other personal care fields. By way of example and not limitation, clothing preferences may be selected or input or provided by a user, such as I purchased shirt X and like this shirt very much—I like the color and fit, but I do not like the collar (too narrow) and color and style of the buttons. This information can be provided by the user at point 1701 and stored at point 1702. At point 1703, the system takes available information as appropriate, such as the jacket being considered, and compares this information with known recommendations and/or rules in point 1704, e.g. clothing preferences, likes, dislikes, etc., and provides provide recommendations and/or necessary information for the user in point 1705, such as “this jacket should work for you in size and style but may be too large in the shoulders.”

Various menus may be provided to guide the user through what he or she has done or encountered. For example, in the food realm, the user may be guided through screens such as dairy, meat/protein, grains, salads, vegetables, fruit, junk food, miscellaneous, etc., Beyond this, for example, if dairy is selected, the user may be directed to yogurt/cottage cheese, milk, cheese, ice cream, etc., and once selected, the user may specify the quantity consumed, e.g. number of ounces or fluid ounces. There may be overlap between menus, such as if a “non-alcoholic beverage” is selected, choices such as water, soft drinks, sports beverage, juice drink, milk, etc. may be provided.

As an alternate, the user may enter a choice, purchase, or relevant information manually, e.g. typing in “6 ounces of kombucha” when kombucha is not offered as a selection. Such an entry may be considered by the system, including but not limited to by a human, the manually entered information classified or categorized, and the system may propagate changes to individual or all devices deployed in the field. For example, the kombucha entry may be determined to be a drink having certain benefits, and may be categorized or considered in relation to the person's digestive and/or overall health, and a kombucha option may be provided for the user, a group of users, or all users and considered in the future.

In the exercise realm, the user may be presented with a question as to the type of activity performed and duration of the activity in some measurable quantity (miles run, time spent, stairs run, etc.) Examples of activities may include but are not limited to walking, running, playing basketball, baseball, hockey, fencing, golf, as well as activities such as gardening, backpacking, mowing lawns, shoveling snow, skipping rope, stair climbing, rowing, weight training, housework, dancing, etc. If desired, such activities and quantities may be converted into a measurement such as calories (believed) expended, based on attributes such as activity, time spent, weight of the user, etc.

Stress values may also be measured, such as the user identifying a particular category or classification of stress, e.g. work, home financial, children, parents, other, as may smoking, e.g. cigarettes, cigars, pipes, vaporizers, hookahs, marijuana, and so forth.

Issues such as headaches can be broken into components for selection/reporting by the user, such as location (front, right side, left side, top, back), intensity (e.g. scale of 1 to 10) and type (dull, sharp, throbbing, numb, gripping, migraine, “worst ever,” etc.) Dizziness may be quantified, such as by vertigo, standing dizziness, lying down dizziness, or otherwise. Vision issues can be quantified, for example, as blurry vision, tunnel vision, floaters, flashing light, loss of vision, or red eye. Infections, ED, and other afflictions may also be quantified or categorized as appropriate.

As may be appreciated from the foregoing including the depiction in FIG. 17, the system may provide recommendations, diagnoses, ask for additional information, or simply collect data without providing any feedback, where all of the foregoing is selected by the user and/or a central server. If someone does not want to receive comment or recommendation, such as for a purchase of a particular cosmetic, he or she can disable this or simply not look at or give any weight to a recommendation or suggestion or diagnosis.

With respect to blood pressure, for example, systolic and diastolic blood pressure may be combined into a blood pressure or “BP” function offered on the top page. Selection of the button may cause a transition to a health page or screen. The system may employ blood pressure cuffs or other appropriate devices used together with an appropriate module to assess blood pressure of the user (SBP, DBP and heart rate may be measured). A time stamp and/or GPS location may be provided. Other screens and other functions may be provided in a similar manner (BMI, etc.)

Thus the current system measures parameters relating to the individual and collects these parameters, either locally or remotely or both, then assesses the parameters, again either locally, remotely, or both, to make a determination about the user and/or a recommendation relating to health, fashion, cosmetics, etc. For example, if the user is interested in purchasing a particular skin cosmetic, she may provide the information of the cosmetic (brand, type, product number, SKU number, or other identifier) and the system may search previously purchased cosmetics, as well as preferences for or issues with previously identified cosmetics, and may indicate such a cosmetic recommendation, such as “not preferred” or “too dark based on previous liked purchases” or “product X is what you should purchase based on past expressed preferences.” The user may be periodically prompted to update preferences. For example, fashions may change, and what the user liked last year may be disliked this year, and he may be consulted about past indicated preferences, particularly when a contrary preference is provided. For example, if a man is interested in purchasing a pair of loafers but had previously only expressed an interest in tie shoes, he may be queried if he prefers loafers, does not have an interest in tie shoes, or prefers something about the loafers in particular.

The interactive diary may be digital and may include artificial intelligence in addition to or rather than rules based decision making, and such artificial intelligence may be embedded in the personal care device or a remote device.

Thus the present system compiles the information using the interactive diary, either passively (without user input) or actively (e.g. by engaging the user, asking questions based on the action or situation presented), assesses the information generally by category, and makes recommendations or provides information to the user, or solicits additional specific information, in either general instances (e.g. displaying a target heart rate during exercise, displaying prior blood pressure readings when taking blood pressure) or specific instances (when the user is considering buying a brown blouse, indicating such a color generally does not match her skin tone, or when queried by the user, suggesting running four days per week instead of two to achieve a desired health/weight goal). Such functionality may include progressing through logic trees or other logic progressions wherein information is assessed and suggestions or information provided for the benefit of the user.

Blockchain Functionality

In addition to the aforementioned interactive diary functionality, the present system employs blockchain functionality to motivate users, as well as track diseases and health issues generally.

The present design employs concepts including disease points (DPs) and health points (HPs). DPs and HPs may be used as part of an insurance plan, for example, where users acquire DPs and HPs based on behavior exhibited and/or incentives provided. DPs and HPs may be distributed and used in other ways, but are generally intended to provide a system for tracking the health of a user and providing incentives for users to enlist appropriate medical care under their specific circumstances. A disease point is issued to the present system, such as to a server arrangement as discussed herein, by an entity such as a government or corporate entity (insurance, etc.). Each DP issues for allocation to users based on the psychological, social, economic and other needs of users in exchange for goods or services from providers or agents, such as web-based agents. In one example, a user may have a need for a liposuction procedure. He may have established an account with a certain number of DPs available, and a facility or medical professional may offer to perform a liposuction procedure for a certain number of DPs. DPs may be provided in a variety of ways, either by payment or allocation from an entity to patients in a particular insurance group or class. DPs can, in some instances, be useful in motivating users toward preventative medicine options.

HPs may be earned by users via a structured reward system for healthy behavior. HPs can be redeemed for benefits, such as goods and services from providers or agents, such as computer based agents. For example, a user may be awarded points and may redeem them for medical examinations or medical related equipment or services, such as exercise equipment, health club memberships, seminars to quit smoking, and so forth. HPs are intended to encourage a positive feedback for healthy behaviors.

DPs and HPs may be stored on the system disclosed herein and may be accessed by users and providers and parties having authorization and an interest, such as insurers. Accounts can be established on the system described herein; a user may have an account that tracks DPs and HPs, as may a provider, such as an insurer, medical provider, merchant, or otherwise.

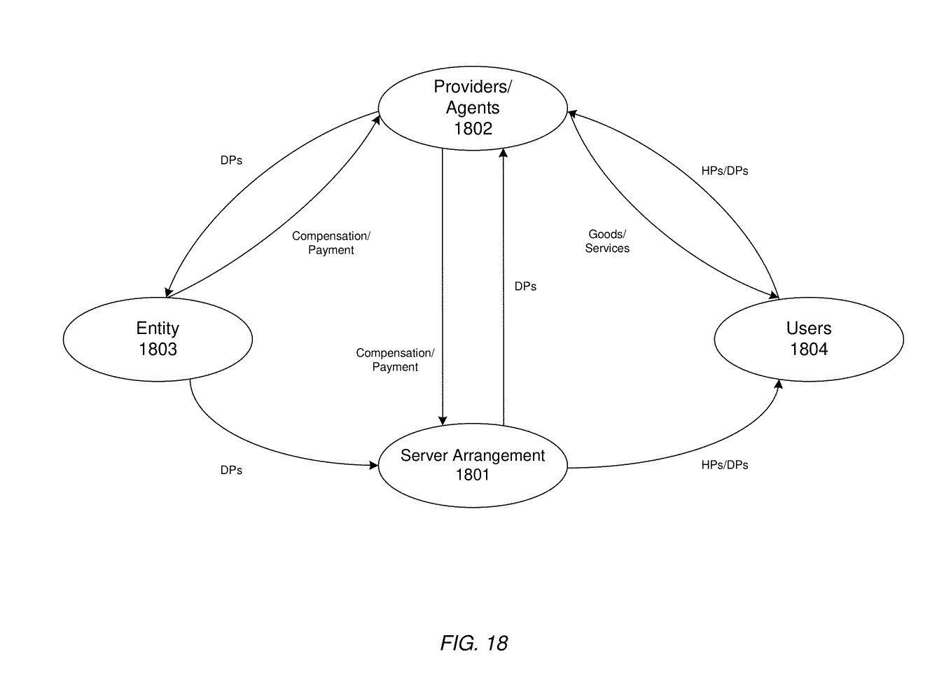

FIG. 18 discloses operation according to this aspect of the design. From FIG. 18, server arrangement 1801 maintains records and accounts for DPs and HPs. The server arrangement manager maintaining or controlling server arrangement 1801 may provide payment or compensation to providers or agents 1802, who may in return provide DPs to server arrangement 1801. Providers or agents 1802 may provide DPs to an entity 1803, such as a government entity or insurance company, and the entity 1803 may provide compensation to the provider or agent 1802. Also shown are users 1804, who may receive DPs from the server arrangement 1801 and goods or services from a provider or providers or agent 1803. Users may provide DPs or HPs to providers or agents 1802. The system, via server arrangement 1801, may provide HPs and DPs to users 1804.

On an encrypted server or server arrangement, a party may maintain transactions involving DPs and HPs using blockchain technology. Basically, blockchain technology is decentralized record maintenance, where a limited number of entities are able to add to the blockchain and once a record is added, it is distributed and available to multiple devices. Some have likened blockchain technology to a ledger distributed across multiple devices. Any wrongdoer attempting to alter records cannot change all records on all devices and thus the distributed nature of the data provides against corruption or malfeasance. The present design employs blockchain technology with DPs and HPs, subject to certain safeguards including authentication and verification. Encryption may be employed. General distribution of records may be limited, and users may not be able to view DPs and HPs of other users. Contract created or written to nodes of the blockchain may be determined by users and providers or agents through a bidding process as described below.

With respect to encryption, for privacy purposes, the information may be encrypted when written to the blockchain. Those having appropriate credentials may be permitted to read the underlying information after decoding or decrypting, while other users coming upon the information without appropriate credentials may simply see encrypted data. Different methods of security and encryption known in the art may be employed such that only those having “a need to know” may be granted access to the underlying information. Alternately, secure information may be kept from the blockchain, and only information such as number of DPs and HPs and/or non-private information may be provided to the blockchain.

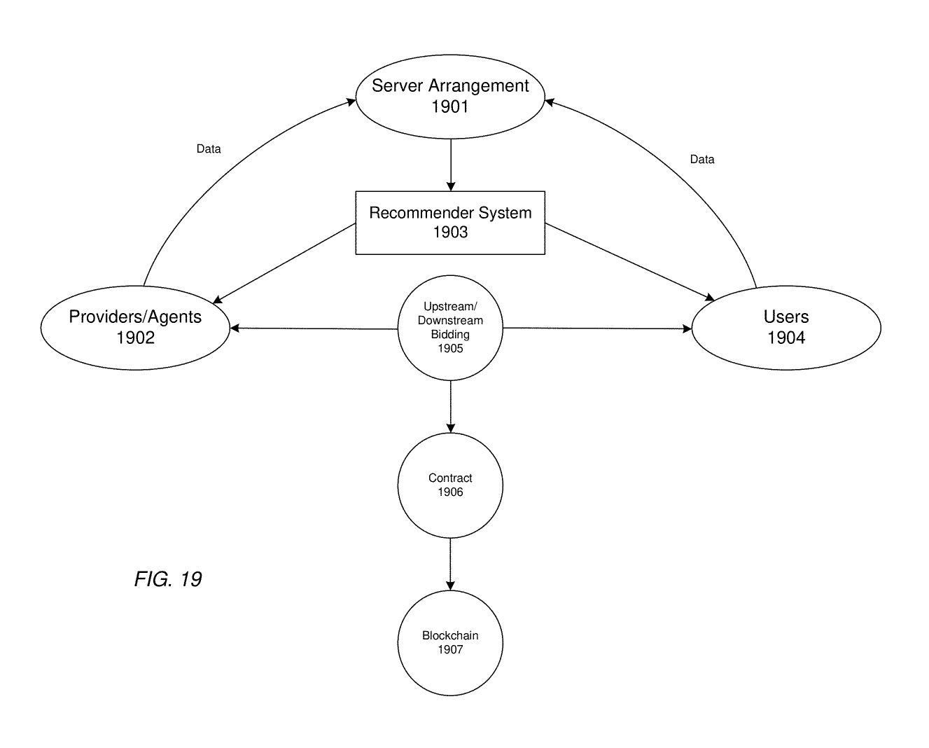

FIG. 19 generally represents contracts and bidding using blockchain technology. From FIG. 19, there is provided a server arrangement 1901 managed by a server arrangement manager and receiving data from providers or agents 1902 and users 1904. Recommender system 1903 receives information from server arrangement 1901 and provides recommendations to providers or agents 1902 and users 1904 as discussed herein. An upstream/downstream bidding module 1905 provides DPs and HPs to users 1904 and providers/agents 1902 based on a bidding system, described below, wherein bids are made and points provided by authorized and controlling persons or entities. Once a bid for DPs and/or HPs is accepted and a contract made, information regarding the contract is transmitted by the controlling entity to the blockchain 1907 in the form of a contract 1906.

Contracts are established and provided to the blockchain when a user agrees to terms with a provider or agent regarding the exchange of goods or services for DPs and/or HPs. Each contract may have a set of conditions and a specified time frame which dictate the null, partial, or full fulfillment of the contract. Every creation or update of a contract is recorded in the blockchain. Contracts may be updated when the determined set of conditions are met or the specified interval of time passes. Users and providers create contracts through the bidding service module or bidding module 1905. This bidding service has two aspects: upstream (from user to provider) and downstream (from provider to user). Users can seek services, goods, and/or recommendations from providers and agents by specifying their health needs. The manager of the server arrangement, for example, may then deliver prospective providers or agents who can offer goods and/or services for these health needs, or they can search for providers or AI agents using the server arrangement 1901. Providers or agents can seek to offer services, goods, and/or recommendations they would like to provide, and the manager of the server arrangement or other entity may deliver prospective clients seeking the services, goods, and/or recommendations to the provider or agent. Alternately, the provider or agent can search for prospective clients using the server arrangement 1901.

Further, using data from providers and users, a recommender system 1903 may match providers and prospective clients. The recommender system 1903 contemplates two specific entities; the user, namely the individual person or agent using the system to give and receive recommendations, and the item, which is whatever item or entity being rated and searched by the user. Users may form a community, which can be a source of information for the database. Other methods of data collection may be employed.

Such a recommender system may employ collaborative filtering, based on the idea that human preferences are correlated, and one individual's preferences can provide a suggestion about another who is similar based on some constraint. Such a system may also employ content-based filtering, which is filtering based on content, and/or knowledge-based filtering. Knowledge based filtering or recommenders query users about what they are seeking, and searches through a database to find selections based on the user's requirements. Knowledge based searchers may ask the user to provide information as to the relevance of the choices offered, and employ learning based on this information.

An agent-based recommendation may also be provided. An agent in this disclosure may represent a person but may alternately include a data miner that locates information about individuals. Though the type of system used to generate site recommendations varies, certain agent recommenders that may be employed include those which use an individual's behavior to provide better suggestions to users within the individual's community (group sharing similar patterns), but also extract implicit knowledge from individual users for the groups to which users belong. Examples of such devices include systems by Zhan et al. (Zhan, Justin; I-Cheng Wang, Chia-Lung Hsieh, Tsan-Sheng Hsu, Churn-Jung Liau, Da-Wei Wang (2008), Towards efficient privacy-preserving collaborative recommender systems, IEEE. pp. 778-783.) and agent-based recommenders of Birukov, Blanzieri, and Giorgini (Birukov, Alexander; Enrico Blanziere, Paolo Giogini (2005), Implicit, ACM, pp. 618-624).

While health and disease conditions are binary, the upstream and downstream bidding system using blockchain can also apply to non-binary conditions were different weights are given to various components of the transaction. For example, in the case of fashion and bidding of a garment, whether upstream or downstream, the different components of the garment, such as material, cut, designer's name, year of design etc. can be given different weights independently by both the provider and the user. These weights are tracked by the blockchain ledger system disclosed herein that can bid the different weights up and down stream for both users and providers until the bid is settled and contract fulfilled. Reward or club points can be part of the different weights of the bidding tender.

The present design may operate in accordance with the following examples. While these discuss operation generally in certain circumstances, such examples are not intended to be limiting or restrictive in any way but are instead intended to provide a general set of circumstances for illustrative purposes only to assist in understanding the overall design.

Assume Patient A is 60 lb. overweight and would like to lose these 60 lb. Patient A has 600 DPs and 100 HPs available to on his system account. Either by searching through a list of providers or agents that may help him lose this weight, or through a recommendation by the system's algorithms, the user determines he would like to use the service of Doctor B. Doctor B offers a liposuction procedure at the price of 100 DPs to Patient A to reduce the weight of Patient A by 60 lbs. Patient A agrees to undergo the procedure at that cost and a contract between Patient A and Doctor B is established and written to the blockchain. Upon completion of the procedure, the blockchain is updated and 100 DPs from Patient A's wallet is transmitted to Doctor B.

Patient P is out of shape and would like to seek the service of a personal trainer. Patient P has 150 DPs and 100 HPs available on his system account, and using the hardware modules disclosed herein, determines he has a BMI (body mass index) of 30%. Patient P bids the amount of 10 DPs and 10 HPs in exchange for a reduction of his BMI by 10%. The system notifies ten personal trainers that there is a prospective client in the area. No one wants to offer their service for Patient P's specified amount, however one personal trainer, Trainer Q, offers Patient P ten 30-minute sessions for 10 DPs and 100 HPs to be used over the next year in order to reduce Patient P's BMI by 10%. Patient P agrees to these conditions, and a contract between Patient P and Trainer Q is established and written to the blockchain. After the year is completed, Patient P and Trainer Q have completed eight sessions and using the hardware modules and system functions disclosed herein, and the system determines Patient P's BMI has decreased by 6%. The system determines how many of the contracted 10 DPs and 100 HPs are transferred from Patient P to Trainer Q, based on terms provided in the original contract between Patient P and Trainer Q. The system updates the contract on the blockchain and the DPs and HPs are transferred.

### Enhanced Telehealth Functionality—Organ Health Assessment

The present design is applicable in the telehealth field, where telehealth contemplates the remote evaluation and management of the health of individuals. The present design enables a healthcare provider to perform a contactless noninvasive physical assessment of a patient or patients using artificial intelligence or other functionality to perform evaluations of various bodily organs and/or body components. Such assessments can provide evaluations yielding reasonable results equivalent to the generally accepted current methods of physical examination (inspection, auscultation, palpation and percussion) but from a remote location.

The present design uses at least one camera, microphone and/or sensor to obtain specific direct measurements including but not limited to intensity of skin coloration, temperature, hair follicle displacement, quivering, fasciculations, constructed and deconstructed multidimensional, directional and non-directional sound waves, spectral gas analysis of emanated body gases, as well as other noninvasive single or multiple data points measurements related to the inferred, predicted and validated body organ vital sign.

The present system thus provides a method of generating a prediction model for body organ health. The system determines vital signs of interest by providing more than one data point source entry with a Model Deconstruction Transfer (MDT) platform comprised of a variable library (VL), wherein each data point enters at least one data set relevant to the vital sign of interest into the MDT platform and selects variables from the VL that are relevant to the vital sign of interest. As an example, when assessing heart health, skin color may be assessed and used as a data point. Such a data point may range in importance anywhere from zero to 100%; the system accounts for the information provided, particularly in conjunction with other information collected, and makes its best attempt to assign weight to the different observations, which may occur over time, and assess health of the patient, particularly his or her organs. Further, the present system may generate at least one prediction model (PMo) for each vital sign from the MDT platform, wherein each PMo is based upon the selected variables. Heart rate, observed pulse, skin color, blood pressure, and so forth may all constitute data points.

The system may also generate a Model Component Library (MCL) from each PMo, wherein the MCL comprises components that result from deconstruction of each PMo. For example, on a Tuesday the system may sense the patient has a skin color, heartbeat, blood pressure, general disposition, and weight. Such readings sensed may be deconstructed and stored in the MCL and subsequent readings may be collected such that the system may at a later point assess patient health, such as organ health. The system may generate at least one second prediction model (PMI) from the MCL, wherein the system employs the at least one PMI to predict the probability of the health vital sign of interest to the healthcare provider for a specific user. As many be appreciated, the system may determine that a user with a current heartbeat of X, blood pressure of Y, smoking history of Z, heartbeats of A1 through A60, representing 60 readings of heartbeat, a blood composition of B, skin color of C, and current body temperature of D may have a risk of stroke of EE, risk of heart attack of FF, and risk of lung cancer of GG. In the system, logic trees and/or other artificial intelligence techniques may be employed. The system may generate a PMI using individual MCL components as variables in the at least one PMI with differential weightings. A single MCL component may be used more than once in a single PMI. For example, a skin color of C may be used in assessing the health of multiple organs or systems in the patient. Further, each use or a single MCL component may be subject to a different statistical limitation. In one instance, skin color may have high importance in assessing and may be assigned a value of, for example, 50%, while in another assessment skin color may have an assessment value of 1%.

The present system further provides a method of generating a prediction model of a vital sign of interest. The system is configured to obtain at least one data set relevant to a vital sign of interest, select variables from each data set relevant to the vital sign of interest, generate at least one first prediction model (PMo) of each vital sign based on differential weighting or at least one data set variable. The system generates at least one PMI and may further generate a Model Component Library (MCL) from each PMI. The MCL comprises components that result from deconstruction of each PMI, and the system may also generate at least one second PMI from the MCL based on a differential weighting, or the MCL components relevant to the vital sign or interest. In one embodiment, the one or more body organ vital signs may be provided by the system to a Model Deconstruction Transfer (MDT) platform comprising a variable library (VL).

In operation, the system may make a number of assessments of a patient at various times. The assessments are done using contactless physical “capture points” from a single device or data stored and exported from other similar devices with historical data entry points. Such capture points may include different readings captured at different points on the patient's body, either during a current test or stored data from previous testing. A patient may have her body temperature, blood pressure, and pulse taken over a period of time, and may have available various blood, urine, and stool samples available by visiting a location wherein the information pertaining to the samples is provided to or received by the system. Further, the system may have available information about the patient, such as age, gender, blood type, known allergies, previous medical procedures performed, and so forth. Information available about the patient collected by the system in these various manners may then be deconstructed to determine relevant attributes and create a prediction model based on this information. For example, if the user has a pulse of 81, the system may build a model for issues when the patient's pulse reaches 90, 100, 70, 60, or other specific values, including risk of stroke, unconsciousness, and so forth. Models may employ multiple patient attributes, primarily know attributes but also including unknown or assumed attributes and attempts to build at least one model predicting a possible course for this patient. A further example may be that information about a patient's heart may be available and based on the information the system may determine that if the patient continues on with his or her current course, he may run an X % risk of having a heart attack or a Y % risk of stroke within the next five years. Such assessments are based on evidence based medical science including statistics and other available relevant information.

Other information may be available, such as information provided by the user, for example representations of his diet or physical activity, mood, stress level, and so forth. The system may have multiple pieces of information relevant to the user available and may employ all information or only certain information in creating prediction models. The MCL may include components from one or more prediction models, such as an expected blood pressure at a point in the future assuming all other actions remain consistent, and such MCL components may be current or forward looking, such as at some point in the future (future white blood cell count, future oxygen intake capacity, etc.) Hence one parameter, such as red blood cell count, may be isolated and used in different models and libraries and may be available for use in prediction or use in a prediction model.

The system may alternately or additionally obtain a further data set from one or more body organ vital signs, wherein this additional data set comprises other body organ vital sign data not represented in the MCL and is thus non-redundant and unrelated to data used to generate the MCL.

This additional data set may include a training set, used for training the system and including either hypothetical or actual attributes, a test set using test information, wherein test information may be used to evaluate hypotheses or models created and typically include previously determined or available data. Alternately, additional data may be provided as a combination of a test set and a training set. Data in a data set may be divided into training and test sets chronologically according to the time of recording.

At least one PMI may include MCL components selected based on performance of the PMI to predict probabilities of the body organ vital sign in the training set or the test set. In such a configuration, when predicting for example heart health and blood pressure, prior blood pressures may be employed in the prediction model, and MCL components may include such prior blood pressures.

The system may generate at least one PMI from the MCL using individual MCL components as variables. In this scenario, the system may use at least one PMI with differential weighting. The system may use a single MCL component more than once in a single PMI. Each use of a single MCL component may be subject to a different statistical function. For example, in one instance criticality of white blood cell count may be zero, in another 20 weighting, in another 50 weighting, and in another 81.3 weighting. Value of the attribute may be a function of other attributes.

A few specific examples are provided. In certain instances, the following examples may be used in the system to validate and predict body organ related vital signs. In the case of heart health, including but not limited to heart rate, blood pressure, cardiac output, vascular resistance, pulmonary circulation pressure, and so forth, the system may take multiple measurements from predetermined body locales, peripheral or axial, either by a healthcare provider or using a 2D or 3D or holographic representation of the user's body, by touching the user's displayed 2D, 3D or holographic image or by the user touching an appropriate part of his or her body with his or her finger. Alternately, the system may determine such parameters using a preliminary scanning data sourcing algorithm that determines a preferred noninvasive data point sourcing from a desired physical point. The physical data sourcing point location is fixed and stored in the system using body surface navigation techniques and retrieved for future reference for data point sourcing.

Regarding representations of the body, such as 2D, 3D or holographic representations, certain technology may be available wherein information about the user's system may be gathered, such as heart attributes, stomach attributes, intestinal attributes, pancreas attributes, circulatory attributes (blood vessels, etc.), bone attributes, brain attributes, and so forth, either by scanning, examination, assumption, or otherwise, and such information is represented in a 2D, 3D or holographic model of the patient, and information may be gathered from this 2D, 3D or holographic model. For example, a patient having undergone heart bypass surgery may have veins removed from his person and inserted in proximity to his heart, and the 2D or 3D model or holographic image may reflect the absence of veins and/or the presence of veins and stitches, etc. near his heart. A patient known to have a brain tumor may have her brain radiographically, anatomically or otherwise represented with the tumor therein such that a remote physician or the system may account for the size, shape, and quality of the tumor. Other representations, such as unknown attributes of the user, may be generic in quality or quantity. For example, a woman with a brain tumor may have no information available about her femur, and a general or generic representation of her femur may be provided for a woman of her size. Information known about the patient may be used in representing unknown organs or body parts. The result is a general model of a patient's body which can be used to assess his or her health in various circumstances and can be modeled to operate in an expected manner based on prior information—for example, heartbeat can be modeled based on known attributes of the heart and prior measurements, such as blood pressure, pulse rate, cardiovascular health, and so forth.

With respect to heart health, in one example, the system uses multiple points of reference, wherein the user may touch one point peripherally on his forearm, one point axially on his face, and one auditory data point related to the organ in question, such as the heart. Data point inputs from a camera, microphone and a sensor may be retrieved where relevant and information gathered is constructed to a single data model. For example, the system may transform sound energy from the microphone to a visual geo-localized three-dimensional image from the “Lub” (systolic) and “Dub” (diastolic) sound wave of the heart. Pixel to pixel convoluted neural network (CNN) analysis of the image may be employed, for example, to render predictive correlations with other heart related parameters not limited to systolic and diastolic blood pressure, stroke volume and cardiac output. Convoluted, recurrent or any other type of software or hardware artificial neural network inspired by biological neural networks may be employed in this process. Reconstructed auditory images of the heart beat can include additional information regarding axis of heart rotation, velocity of blood column, valvular auditory inputs, blood viscosity, electrical signal spread in the bundle of his or other heart beat signal attributes. Additional readings from distant areas may also augment heartbeat information, with the visible pulsing of veins and arteries under the skin of the user. Although data is received as different forms of energy, such as sound energy versus light or visual energy or other readings, the system transforms differently sourced data points (one or more) to one uniformly comparable data format. The system compares and correlates the different data sources to a referenced vital sign, such as pulse or blood pressure. The measurements taken by the system can be recorded and used to train the user specific model of the individual patient to verify or validate a predicted blood pressure. The system may also test the user specific AI model to fine tune prediction accuracy. The system may employ attributes of a patient, including attributes retrieved, calculated, and/or predicted, with other patients by matching attributes between patients, including but not limited to genetic and physical attributes of individuals. The system compares a predicted vital sign value with the actual measured vital sign and may correct for the difference by re-adjusting the different weights previously rendered, continually fine tuning error margins and narrowing the gap between actual and predicted values.

For example, if patient X has a certain BRCA genetic makeup and developed breast cancer at age Q, and patient Y has a similar physical makeup and BRCA gene distribution or profile, Patient Y may be expected, with a level of uncertainty, that she may contract breast cancer at a similar age. In this manner, the system may obtain, calculate, or predict other heart related vital signs that can be evaluated and/or correlated to diagnose and manage heart diseases, for example.

In the case of lung health, including but not limited to respiratory rate (RR), CO2 level, lung volume, pulse oximetry, breathing characteristics, jugular vein distension (JVD), pitting edema, and/or body pH level, the same general overall concept applies. The system may obtain oxygen saturation via pulse oximetry (SaO2) by the user directly putting his or her finger on a body location collected using a camera in addition to noninvasively obtain the lung sounds at apices or bases of the lungs. The system collects or correlates such measurements with chest wall expansion using two observation points on the exterior of the patient or chest wall spatial excursion. The system, from this information, may determine a value, known as the FEV1/FVC ratio or Tiffeneau-Pinelli index used in the diagnosis and management of restrictive and obstructive lung diseases. The system may obtain other lung related vital signs and may correlate collected information for the diagnosis and management of lung diseases. For example, as described herein, the system may use a microphone, camera, and other components, including but not limited to other medical sensing mechanisms to sense attributes of the patient, either visually available, audibly available, or otherwise available to make the determinations and assessments provided herein.

The system may also determine vascular health. Vascular Health in this sense may include, but is not limited to, vessel pliability, temperatures of hands or feet, hair loss, ulceration, atrophy, and/or skin discoloration. The system may employ a camera to measure the degree of two or multi points displacement of, for example, hair follicles due to superficial pulsations of a midsize artery like the radial artery at the wrist, or the patient's superficial temporal artery in the temple area, or a large size artery such as the carotid artery in the neck. The system may correlate these visual data points with visually transformed images of heart sounds, and in this manner the system can establish a vascular wall elasticity index. Such an index may facilitate the diagnosis and management of vascular disease.

The system may also assess, predict, and diagnose issues with skeletal health, including but not limited to deformities, warmth, swelling, range of motion (ROM), and/or presence of scoliosis. The system noninvasively assesses sounds emanating from joints and correlates such sounds to visual cues from specific joints e.g. range of motion, deformities, warmth, and swelling. As a result, the system establishes a flexibility index used in the diagnosis and management of each body joint health and disease state.

In the area of gastrointestinal microbiota health, the system noninvasively assesses sounds and gases emanating from the patient's gastrointestinal tract and correlates such sounds and gases to an axial and peripheral skin perfusion color analysis as well as heart sound. The system indirectly assesses gastrointestinal perfusion and generates and follows a digestive index that can be used to assess the gut microbiota and their response to different foods or pharmaceuticals, as well as ingestible items that can be avoided, and any change in the gut microbiota that may result or influence the gastrointestinal health or disease states.

The system may auditorily receive and assess bowel sounds, where such sounds represent the intrinsic gastrointestinal secretions as well as gas produced by gut microflora or microbiome. The gas content (echo chamber) of the bowel reflects the metabolic activity of the gut bacteria (microbiome) and such metabolic activity is reflected in the heart rate and blood pressure of the patient via the gastrointestinal—brain connection. The immune system is also involved in this delicate dance with fluctuation of body temperature related to “capillary gating” that allows nutrients as well as chemicals and some bacteria or their byproducts to cross over to the blood stream, causing some antibody immune response. The system, monitoring bowel sounds and other available information, may provide a recommendation such as “adjust your carbohydrate intake by 50 grams per day, to avoid gas distention and increase in your heart rate” or “your temperature was elevated following this protein rich meal and an immunoglobulin blood assay test was ordered to be done with in the next 30 minutes,” with blood recommended to be drawn, such as by a health band, and analyzed immediately or at a later time.”

The system may also assess sleep architecture health. Healthy adults typically need between 7 and 9 hours of sleep per night to function at their best. Most of the brain reparative processes happen during sleep. Sleep architecture refers to the basic structural organization of normal sleep. Two types of alternating sleep cycles exist, non-rapid eye-movement (NREM) and rapid eye movement (REM). Irregular cycling and/or absent sleep stages are associated with sleep disorders. Cardiovascular, respiratory, sympathetic, renal and endocrine body stems show physiologic changes that occur during sleep. While a user is asleep, the system may assess sounds of turning on bed and degree of snoring or breathing, and these sounds can be correlated with heart rate and other visual cues to noninvasively assess and follow the sleep architecture health.

The present system may seek to statistically “curve fit” and compare predicted data points using known predictive analytic techniques to actual data points. The system also employs contextual adaptation techniques to narrow the gap in curve fitting between multiple, such as three, elements of prior historical statistical analysis, current real time data points and predicted future values of data. The present system uses synthetic control of data points with “go/no go” methodology that allows data points above a certain predetermined weight to enter into the assessment and recommendation process described herein.

The present system thus obtains, assesses, and predicts body organ vital signs noninvasively by combining visual, auditory and other sensory data sensed from a device, such as a device having a camera, microphone, and access to other relevant medical assessment equipment, potentially joined to the system. The system may also receive patient data from other sources, such as external sources at single or multiple “capture point(s),” and may determine information about organ health from peripheral data or statistics. The system also tracks body organ health and disease states remotely and noninvasively using a device such as the reflective surface device described herein and/or a portable handheld device such as that discussed. The user may enter information—foods consumed, mood, general health, specific health, via the devices presented herein. The present design thus provides a personal care module that is configured to receive, from a sensor configured to detect blood distribution analysis and spread at two or more regions of a user via user skin color, tone, temperature, condition (dry/soft/firm/swollen/sunken), and capillary refill. The system may, in this instance, receive an image indicating a first blood distribution and spread for a first region and another image indicating a second blood distribution and spread for a second region. The system may compare the first blood distribution and spread for the first region and the second blood distribution and spread for the second region to provide a blood-distribution/spread asymmetry representing a difference in heart health between the first region and the second region.

Alternately, the system may receive and compare data from a microphone sensor capable of detecting body organ sounds. Data is captured through a microphone sensor targeted specifically and compared to the same physical point location of a body region on the patient. The system compares capillary refill, with different data obtained from different sensors (optical, infra-red, tactile, etc.) as well as a microphone to indirectly or directly infer and determine respiratory rate (RR), lung volume, pulse oximetry, breathing characteristics, jugular vein distension (JVD), and/or body pH level, pulse/heart rate, blood pressure, heart rhythm, and EKG values using appropriate hardware and hardware modules. The device may collect, compare, and measure data either directly or using processing to directly or indirectly infer a body organ measurement from a baseline measurement. As such, the system assesses body organ health, and generates a specific health suggestion to the user in one embodiment.

Alternatively, the system may receive and compare data from a device such as a gas spectral analysis sensor capable of detecting different gases emanating from body orifices or skin. The gut—brain axis is a communication system that integrates neural, hormonal, and immunologic signaling between the gut and the brain and other organs, offering the intestinal microbiota a potential route to access and influence multiple body organs. The gut microbiota is implicated in nutrient acquisition and energy harvest and produces exo-metabolites, such as short chain fatty acids (SCFAs), that may regulate a host metabolic process. Gas chromatography-mass spectrometry or other means can noninvasively assess both mouth odors from halitosis or fecal gases not limited to butyrate. Oral, gastrointestinal and related organs like tongue, salivary glands, liver and spleen can be remotely and ono invasively assessed. The system may assess these factors or characteristics and may provide health suggestions by applying the current methodology.

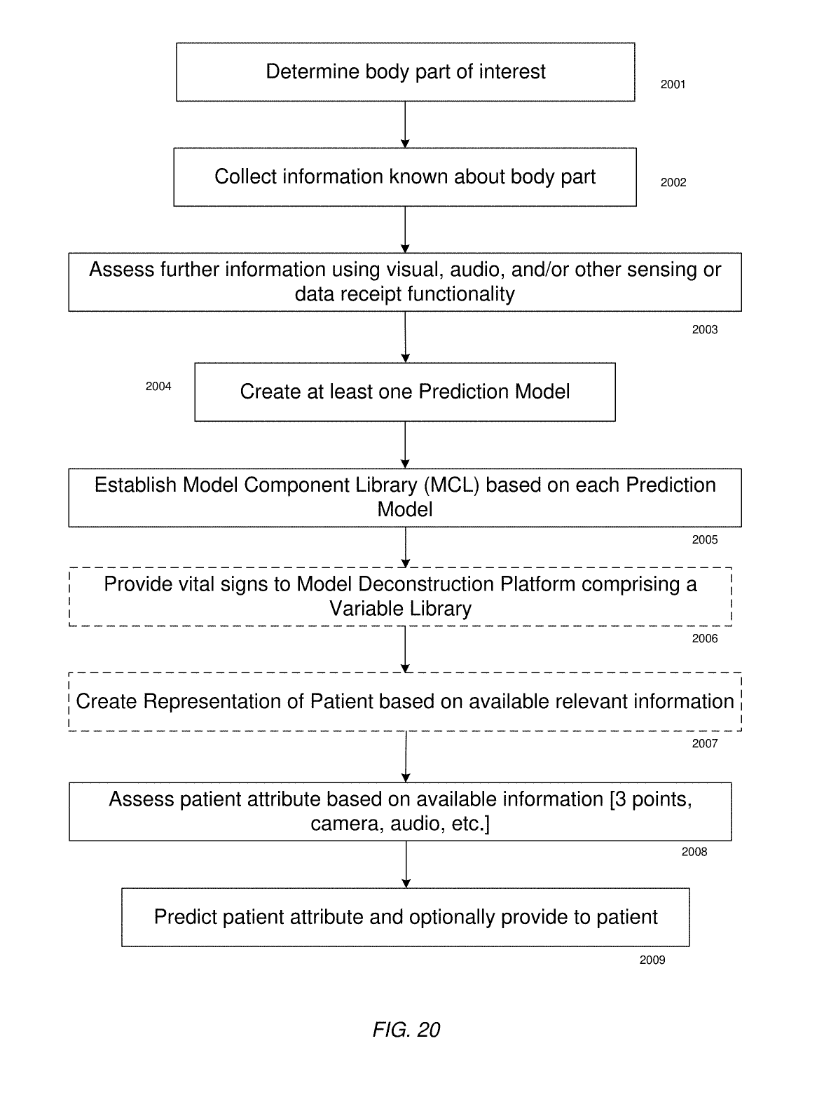

FIG. 20 illustrates a general representation of operation according to the present design. From FIG. 20, point 2001 employs the device to determine a body part of interest, e.g. right hip, heart, pancreas, brain, left ankle, right lung, etc. Point 2002 collects information known about the body part, either previously assessed or predicted or calculated. At point 2003, the system assesses further information using the system hardware disclosed herein, including video, audio, and/or other sensing functionality. Point 2004 represents the system creating at least one prediction model based on data collected, while point 2005 establishes a model component library based on data contained in relevant prediction models. Point 2006 is optional and includes the system providing vital signs to Model Deconstruction Platform, which deconstructs the model into component parts, where the Model Deconstruction platform includes a Variable Library representing variables applicable to various patient attribute assessments and predictions. Point 2007 is also optional and represents the system creating or enhancing a representation of the patient based on relevant information, wherein the representation may be 2D, 3D, holographic, or even textual or any other form of patient representation known in the field. Point 2008 is the system assessing at least one patient attribute based on available information, such as visual determinations from multiple points of the user's body, visual determinations and audio determinations, etc. At point 2009, the system may predict patient attributes or a single attribute and may optionally provide the prediction to the patient. Again, such predictions depend on circumstances but may be as mundane as “if you do not take aspirin, you run the risk of further wrist pain” to “you may be at elevated risk of cancer in less than 8 years.”

Individual or collective vital readings, organ assessment and or recommendations employ the blockchain concepts disclosed herein, i.e. are provided on a distributed ledger format, to aid different health providers to review, assess or retrieve information for further clinical management, third party billing, or other administrative related activities. Blockchain provides regular, such as daily, reporting that includes entries of desired categories (e.g. food, beverage, activities, symptoms, medicine and vitals) in addition to recommendations, wherein recommendations are determined either via artificial intelligence or provider interactions, and/or recommendations for a specific date of service.

The system may self train. In one example, if today's measured blood pressure is 140/90 the system accounts for all available information such as calories, symptoms, medications, activity, weather, season, GPS location, genetic factors and other factors, and may predict a number for blood pressure at a specific time the following day. The patient measures his/her blood pressure on that specific time the following day. The system accounts for any difference between actual and predicted measurements and creates a training set of systolic and diastolic numbers to adjust future prediction accuracy. Such test/correct processing continues as long as necessary to lower the probability of a successful reading to as low a value as possible. For other vital signs, such as heart rate, readings can be assessed using available devices such as a health tracker or other peripherals, or contactless in some situations.

The system may call for a blood sample at certain times, wherein blood drawn may be analyzed instantly or later to train the system further. Training can include radiological, laboratory or other diagnostic data. The system obtains patient history, performs a physical exam, and orders tests. The system then diagnoses based on known information to be able to recommend different management options. The system may employ auditory sensing to receive a conversation between doctor and patient, and may assess doctor textual entries to make recommendations.

In general, during operation, the user/patient positions himself/herself in front of the device and/or sensors, or uses the modules available. The user points to area of symptomatology. Sensors focus on this area, determined by sensing the user finger and the region proximate the tip of the finger, and gathers visual data noninvasively. Sensors may gather data from other areas of the body. The system may determine or call for other relevant sensor modalities to gather further information from the area in question or other related areas. The data gathered may be represented textually, or in a 2D, 3D, or holographic representation of the individual (heart size, bone attributes, brain attributes, etc.), and/or may be represented on a holographic avatar. At this point, the system may initiate processing, by consulting with known information and/or recommendations, and may determine and present diagnostic recommendations. Such recommendations may be provided to the patient, insurance representative, or other authorized individual or entity, and desired milestones may be provided or assessed. The system may then create a blockchain record. The user/patient may be incentivized at each agreed upon milestone with health crypto-currency or other incentives.

In the specific case of obesity and depression, the user/patient positions himself or herself in front of the device and/or sensors. The sensors gather auditory data from gastrointestinal tract, as well as heart rate, skin perfusion, user voice and an emotional analysis, which may be administered by a series of questions or may be based on factors such as posture, expression, and so forth. The system predicts future weight and a future depression score, and these may be provided to the patient. The system may offer communication capability for the user to communicate regarding his or her predictions with the system or a professional. The system may then, based on knowledge of the medical fields of obesity, diet, nutrition, etc., develop a set of diagnostic recommendations. Milestones can be planned, discussed, and/or approved by a provider or insurer, as well as with the patient/user and other third party. The system may provide to the user an incentive methodology incentivizing change in patient behavior, and such may be approved by an insurer or other third party, or by the provider, and the patient/user. The system may create and distribute a blockchain record.

One additional attribute of the present design is that during a provider—user interactive session (or sessions), data sources from different audio, visual or other data input(s) can be transformed into a unified data source and be processed as such. For example, the conversation between a provider and a user can be transcribed to visual text and the system can weigh symptoms shared by a user based on speech and facial expression sentiment analysis. Spoken words by both user and provider in this example are transformed to one or multiple 2D, 3D, or holographic visual representations to be entered in “go/no go” data processing for the specified body organ. The system can search and retrieve the unified data source in the transformed or original format(s) as to facilitate bodily organ health assessment(s).

Additionally, in the specific area of neurological and mental health, speech analysis is not necessarily limited to sentiment, anxiety, irritability, contentment, aggressiveness or other speech dysfunctionalities and may include dysarthria, dysphasia, staccato, and/or scanning type of speech. The system can analyze such speech attributes by transforming sound waves of the conversation between the user and provider into 2D, 3D, or holographic visual representation(s). Correlated image(s) or video of pupillary dilatation or constriction, degree of sweating, rate of eye blinking, eye movement, motor strength, balance, coordination, sensory perception, cranial nerves function, degree of alertness, ability to perform calculations, spatial correlations, higher executive brain functions as well as user/patient's temperature, heart rate, respiratory rate, tremors, fasciculations, quivering or other visual imageries of the user would be entered into pixel to pixel convoluted, recurrent or any neural network analyses of the unified image(s) data source(s). For example, the system may employ such speech/physical attributes to render predictive correlations related to “brain health.” The system may generate other forms of indices, for example an “organ response to treatment” index, “organ disease burden” index, and “social burden” index. The “social burden” index can be an indicator of impact on family members, providers, or society as a whole. The system may generate, correlate, assess, and provide other types of indices related to organ health or the lack thereof.

Thus the present design may include an apparatus comprising a sensor configured to optically detect blood distribution analysis and spread at at least two external regions of a user via at least one of skin color, skin tone, skin temperature, skin condition, and capillary refill, and a processor configured to determine an image based on the optical detection of the sensor, the image indicating a first blood distribution and spread for a first region of the user and a second image indicating a second blood distribution and spread for a second region. The processor compares the first blood distribution and spread for the first region and the second blood distribution and spread for the second region to provide a blood-distribution/spread asymmetry representing a difference in heart health between the first region and the second region.

According to a further embodiment, there is provided an apparatus comprising a microphone configured to receive audio data including body organs sounds of a patient at a first point of the patient's body, wherein the audio data comprises capillary refill audio data, and a processor configured to compare the audio data received with audio data previously recorded at the first point of the patient's body and with different data obtained from a different non-audio sensor and determine at least one of respiratory rate (RR), lung volume, pulse oximetry, breathing characteristics, jugular vein distension (JVD), and/or body pH level, pulse/heart rate, blood pressure, heart rhythm, and EKG values.

According to another embodiment, there is provided an apparatus comprising a personal care display device comprising a plurality of sensors configured to sense attributes of a patient, wherein the plurality of sensors comprises a video image collection device and an audio collection device, and a remote central server device arrangement connected to the personal care device. The personal care display device is configured to receive information from the patient, including receiving multiple pieces of information from the video image collection device from the exterior of the patient, compile information about the patient and determine organ health of the patient based at least in part on the multiple pieces of information received from the video image collection device.

The devices, processes and features described herein are not exclusive of other devices, processes and features, and variations and additions may be implemented in accordance with the particular objectives to be achieved. For example, devices and processes as described herein may be integrated or interoperable with other devices and processes not described herein to provide further combinations of features, to operate concurrently within the same devices, or to serve other purposes. Thus, it should be understood that the embodiments illustrated in the figures and described above are offered by way of example only. The invention is not limited to a particular embodiment, but extends to various modifications, combinations, and permutations that fall within the scope of the claims and their equivalents.

The design presented herein and the specific aspects illustrated are meant not to be limiting, but may include alternate components while still incorporating the teachings and benefits of the invention. While the invention has thus been described in connection with specific embodiments thereof, it will be understood that the invention is capable of further modifications. This application is intended to cover any variations, uses or adaptations of the invention following, in general, the principles of the invention, and including such departures from the present disclosure as come within known and customary practice within the art to which the invention pertains.

The foregoing description of specific embodiments reveals the general nature of the disclosure sufficiently that others can, by applying current knowledge, readily modify and/or adapt the system and method for various applications without departing from the general concept. Therefore, such adaptations and modifications are within the meaning and range of equivalents of the disclosed embodiments. The phraseology or terminology employed herein is for the purpose of description and not of limitation.

What is claimed is:

1. An apparatus comprising:
a sensor configured to optically detect blood distribution analysis and spread at at least two external regions of a user via at least one of skin color, skin tone, skin temperature, skin condition, and capillary refill; and
a processor configured to determine an image based on the optical detection of the sensor, the image indicating a first blood distribution and spread for a first region of the user and a second image indicating a second blood distribution and spread for a second region;
wherein the processor compares the first blood distribution and spread for the first region and the second blood distribution and spread for the second region to provide a blood-distribution/spread asymmetry representing a difference in heart health between the first region and the second region.

2. The apparatus of claim 1, wherein the processor is further configured to collect audio data of bodily functions related to the user and use collected audio information to assess organ health of the user in conjunction with data collected from the sensor.

3. The apparatus of claim 2, wherein the audio data comprises blood flow sounds, and the system determines organ health based on the blood flow sounds and optical representations of the first region and second region of the user.

4. The apparatus of claim 1, wherein the apparatus is configured to receive additional information comprising at least one of information provided by the user, information provided by a third party, and information provided by a remote device, and the processor is configured to employ the additional information with the first blood distribution and spread and the second blood distribution and spread.

5. The apparatus of claim 1, wherein the apparatus further comprises an audio sensor and the audio sensor is configured to receive bodily movement sounds and the processor is configured to determine skeletal health of the user based on the bodily movement sounds.

6. The apparatus of claim 1, wherein the processor produces a prediction model based on information received and predicts user health based on information received.

7. The apparatus of claim 1, wherein the apparatus creates a representation of the user based on data sensed.

8. An apparatus, comprising:
a microphone configured to receive audio data including body organs sounds of a patient at a first point of the patient's body, wherein the audio data comprises capillary refill audio data; and
a processor configured to compare the audio data received with audio data previously recorded at the first point of the patient's body and with different data obtained from a different non-audio sensor and determine at least one of respiratory rate (RR), lung volume, pulse oximetry, breathing characteristics, jugular vein distension (JVD), and/or body pH level, pulse/heart rate, blood pressure, heart rhythm, and EKG values.

9. The apparatus of claim 8, wherein the apparatus is configured to collect, compare and measure data either directly or by using artificial intelligence algorithms to directly or indirectly infer a body organ measurement from a baseline measurement.

10. The apparatus of claim 8, wherein the processor is further configured to collect visual representations of external manifestations related to the patient and use collected visual information to assess organ health of the patient in conjunction with data collected from the microphone.

11. The apparatus of claim 8, wherein the apparatus is configured to receive additional information comprising at least one of information provided by the patient, information provided by a third party, and information provided by a remote device, and the processor is configured to employ the additional information with the audio data collected to assess organ health of the patient.

12. The apparatus of claim 8, wherein the audio sensor is configured to receive bodily movement sounds and the processor is configured to determine skeletal health of the patient based on the bodily movement sounds.

13. The apparatus of claim 1, wherein the processor produces a prediction model based on information received and predicts patient health based on information received.

14. The apparatus of claim 1, wherein the apparatus creates a representation of the patient based on data sensed.

15. An apparatus, comprising:
a personal care display device comprising a plurality of sensors configured to sense attributes of a patient, wherein the plurality of sensors comprises a video image collection device and an audio collection device; and
a remote central server device arrangement connected to the personal care device;
wherein the personal care display device is configured to receive information from the patient, including receiving multiple pieces of information from the video image collection device from the exterior of the patient, compile information about the patient and determine organ health of the patient based at least in part on the multiple pieces of information received from the video image collection device.

16. The apparatus of claim 15, wherein the personal care display device comprises a processor configured to collect information known about a body part of the patient, assess all information available including visual and audio information, create at least one prediction model, and assess a patient attribute based on all available information.

17. The apparatus of claim 16, wherein the processor is further configured to predict one patient attribute based on all information collected about the patient.

18. The apparatus of claim 15, wherein the personal care device comprises a plurality of connection ports, each connection port configured to receive a hardware personal care module.

19. The apparatus of claim 15, wherein the personal care device is configured to assess at least one of organ health and skeletal health based on data received from the video image collection device and the audio collection device.

20. The apparatus of claim 15, wherein the personal care device is configured to receive additional information comprising at least one of information provided by the user, information provided by a third party, and information provided by a remote device, and employ the additional information with all other information available to determine a health attribute of an internal component of the user's body.
